(19) United States
(12) Patent Application Publication
(10) Pub. No.: US 2026/0101861 A1
(43) Pub. Date: Apr. 16, 2026

(54) TOBRFV RESISTANT TOMATO PLANT COMPRISING A MODIFIED CCA GENE

(71) Applicant: RIJK ZWAAN ZAADTEELT EN ZAADHANDEL B.V., De Lier (NL)

(72) Inventors: Jonathan KALISVAART, De Lier (NL); Raoul Jacobus Johannes Maria FRIJTERS, De Lier (NL); Alwin Johannes Marinus ROOVERS, De Lier (NL); Daniël Johannes Wilhelmus LUDEKING, De Lier (NL)

(21) Appl. No.: 19/306,232

(22) Filed: Aug. 21, 2025

Related U.S. Application Data
(63) Continuation of application No. 18/162,075, filed on Jan. 31, 2023, now abandoned.
Application No. 18/162,075 is a continuation of application No. 18/156,114, filed on Jan. 18, 2023.

(51) Int. Cl. A01H 6/82; A01H 1/00; A01H 5/08

(52) U.S. Cl. CPC A01H 6/825; A01H 1/126; A01H 5/08

(57) ABSTRACT

The present invention relates to a modified CCA gene which encodes a CCA-adding enzyme, which modified CCA gene leads to resistance against a positive-strand RNA virus having a TLS, wherein the modified CCA gene comprises a gene comprising a nucleotide sequence that encodes a CCA-adding enzyme, a gene comprising a promoter sequence, or a gene comprising a nucleotide sequence that encodes a CCA-adding enzyme having a deletion, a substitution, or an insertion of at least one amino acid. The invention further relates to plants and seeds comprising the modified genes, methods for making and identifying such plants and use of the gene.

### RELATED APPLICATIONS AND INCORPORATION BY REFERENCE

This application is a continuation of U.S. application Ser. No. 18/162,075 filed Jan. 31, 2023, which is a continuation of U.S. application Ser. No. 18/156,114 filed Jan. 18, 2023.

The foregoing applications and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Jan. 18, 2023, is named Y7954-00562SL.xml and is 37,733 bytes in size.

### FIELD OF THE INVENTION

The present invention relates to a Solanum lycopersicum plant comprising a modified gene which leads to resistance against Tomato Brown Rugose Fruit Virus (ToBRFV). The invention further relates to methods for producing such a plant, methods for identification of the modified gene, and methods for selection of a ToBRFV resistant plant comprising the modified gene. The invention also relates to a marker for identification of the modified gene in a plant, and to use of said marker.

### BACKGROUND OF THE INVENTION

In 2015 the occurrence of a new tobamovirus in tomato was published (Salem et al: A new tobamovirus infecting tomato crops in Jordan. Arch Virol. 2016 February; 161(2):503-6. Epub 2015 Nov. 19). This virus was shown to be related to the known tobamoviruses Tobacco mosaic virus (TMV), Tomato mosaic virus (ToMV), and Tomato mild mottle virus (ToMMV), with sequence identities of around 80% to 90% for the closest related sequences of ToMMV and ToMV. Symptoms were rather mild on the plant, but very severe brown rugose symptoms were present on almost all fruits. The virus was observed to break the resistance of the commonly used resistance genes against ToMV: Tm-1, Tm-2, and Tm-2², which is also known as Tm-2^{a}. A later publication showed that the virus was also found in Israel, and it was established that the virus can also infect pepper (Capsicum annuum) plants (Luria et al (2017): A new Israeli tobamovirus isolate infects tomato plants harboring Tm-2² resistance genes. PLoS ONE 12(1):e0170429. Doi:10.1371/journal.pone.0170429). Symptoms appeared to vary based on the affected variety, and in certain instances symptoms were mainly found on the vegetative parts in the form of severe or mild mosaic, necrosis, leaf distortion, or other symptoms. As the virus was clearly different from the known tobamoviruses it was described with a new designation: Tomato brown rugose fruit virus (ToBRFV).

The virus is at least transmitted mechanically, which makes the spread easy and rapid, and difficult to control. Transmission of the virus is also likely to occur through infected seed. This very effective transmission resulted in a rapid spread of the virus to many regions, and the disease caused major problems for tomato production in certain areas. The severity of the fruit symptoms leads to a huge impact of the presence of ToBRFV, since it makes the fruits basically unmarketable. The urgency to obtain resistant tomato plants was therefore very high, and an extensive germplasm screen was organized to determine the possible existence of resistant sources.

Citation or identification of any document in this application is not an admission that such document is available as prior art to the present invention.

### SUMMARY OF THE INVENTION

It is an object of the present invention to provide a tomato plant of the species Solanum lycopersicum that comprises a modified gene which leads to ToBRFV resistance.

Solanum lycopersicum has various wild relatives that harbor disease resistances and are a valuable resource for breeding. Many of the latest tomato varieties therefore already possess one or more introgressions from wild species. However, it appeared that presently cultivated tomato varieties, including the ones that already have tobamovirus resistance genes from wild relatives, were easily infected by this new virus. It was therefore anticipated that it would not be straightforward to identify resistance.

Surprisingly, after extensive screening, three accessions of the species Solanum pimpinellifolium could be identified that were highly resistant to ToBRFV. A research program was subsequently set up to determine if the resistance could be transferred to Solanum lycopersicum, and to identify the genetics behind the resistance.

Crosses were made between the three S. pimpinellifolium sources GNL.3919, GNL.3920, and GNL.3951 on the one hand, and internal breeding lines on the other hand. These initial crosses were followed up with population development, such as F2's, F3's, and backcross populations, for QTL mapping. On all generations bio-assays were carried out to confirm and monitor the resistance in the various populations, and to determine the inheritance (Example 1). The identification and characterization of a QTL through molecular markers gives the opportunity to use genetically linked markers to identify the presence of the QTL, and therefore the presence of the resistance, which is more efficient than the use of a bio-assay.

For this purpose QTL mapping studies were performed. A first QTL mapping on F2 populations resulted in the identification of a QTL region on chromosome 11 that was present in populations that were developed based on all three S. pimpinellifolium sources. However, to work with the resistance even more efficiently and accurately, it is crucial to determine the actual causal gene within the QTL that leads to the resistance. Finemapping of the identified regions was therefore subsequently carried out, followed up with detection of the causal gene (Examples 2 and 3).

The present invention provides a Solanum lycopersicum plant comprising a modified CCA gene on chromosome 11 that leads to Tomato Brown Rugose Fruit Virus (ToBRFV) resistance. The modified CCA gene is in particular a gene originating from, or introgressed from, the species S. pimpinellifolium.

Numerous genes have been recognized for their involvement in virus resistances in plants. However, for a large number of viruses, no resistance gene has been identified yet. Especially for relatively new viruses, or viruses that are similar to others but breaking through known resistances, there is always the challenge to identify a new source of resistance before the virus damage becomes too extensive. Newly identified resistance genes can also be beneficial for the protection of crops against already known viral diseases.

The finemapping of the chromosome 11 QTL region from the S. pimpinellifolium sources in S. lycopersicum populations that segregated for ToBRFV resistance revealed a small region on chromosome 11 that contained only four potential genes which were likely to contribute to the ToBRFV resistance. Whole genome sequences were available in-house for the backgrounds of the resistant and susceptible lines that were used in the development of these populations. A SNP-calling approach for the region was done, which means unique polymorphisms in the region were identified through comparing the sequences to each other (Example 3).

Among the genes in the region of interest were two CCA genes, which were designated CCA1 and CCA2. Various polymorphisms between the CCA1 gene of susceptible and resistant material were identified. The CCA2 gene also contained various polymorphisms, but in all lines, including the susceptible material, the CCA2 gene had an early stop codon which resulted in a truncated protein that did no longer contain all essential active sites of a CCA-adding enzyme. The encoded protein was truncated within the polyA_pol_C-terminal region-like domain, and as a result only the first of three active sites of this domain is still present in the CCA2 gene of S. lycopersicum, as well as in the CCA2 gene of the S. pimpinellifolium sources (FIG. 4).

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### Deposits

Seed of tomato Solanum lycopersicum comprising a modified CCA gene of the invention on chromosome 11 homozygously, was deposited with NCIMB Ltd, Ferguson Building, Craibstone Estate, Bucksburn, Aberdeen AB21 9YA, UK on Sep. 11, 2017 under deposit accession numbers NCIMB 42882, NCIMB 42885, NCIMB 42887, and NCIMB 42890.

Seed of tomato Solanum lycopersicum comprising a modified CCA gene of the invention homozygously was deposited with NCIMB Ltd, Ferguson Building, Craibstone Estate, Bucksburn, Aberdeen AB21 9YA, UK on 7 November, 2019, under deposit accession numbers NCIMB 43511 and NCIMB 43512.

The Deposits with NCIMB Ltd, under deposit accession numbers NCIMB 42882, NCIMB 42885, NCIMB 42887, NCIMB 42890, NCIMB 43511, and NCIMB 43512 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1—CDS sequences of SEQ ID NO: 1 (the wildtype CCA1 gene of Solanum lycopersicum) and SEQ ID NO: 5 (the wildtype CCA2 gene of Solanum lycopersicum).

FIG. 2—protein sequences of SEQ ID NO: 2 (the wildtype CCA-adding enzyme encoded by SEQ ID NO: 1), SEQ ID NO: 7 (the wildtype CCA-adding enzyme encoded by SEQ ID NO: 5), and SEQ ID NOS: 8-11 (CCA-adding enzymes with modifications that lead to resistance). CCA1_NCIMB 43511 and CCA1_NCIMB 43512 are the same as SEQ ID NO: 8. CCA1_TO1 is the same as SEQ ID NO: 9. CCA1_Ramyle F1 and CCA1_S13_00 are the same as SEQ ID NO: 2. CCA2_NCIMB 43512, CCA2_S13_00, CCA2_Ramyle F1, and CCA2_Endeavour F1 are the same as SEQ ID NO: 7. TO1 is the same as SEQ ID NO: 11.

FIG. 3a—promoter sequences of SEQ ID NO: 3 (promoter of the wildtype CCA1 gene of Solanum lycopersicum), SEQ ID NO: 17 (promoter of the wildtype CCA2 gene of Solanum lycopersicum) and SEQ ID NOS: 12-16. CCA1_NCIMB 43511_43512 is the same sequence as SEQ ID NO: 12. FIG. 3b shows an alternative alignment of a stretch before nucleotide 917, which stretch comprises a deletion in all of these sequences when compared to SEQ ID NO: 3.

FIG. 4—representation of the domains of the CCA-adding enzyme.

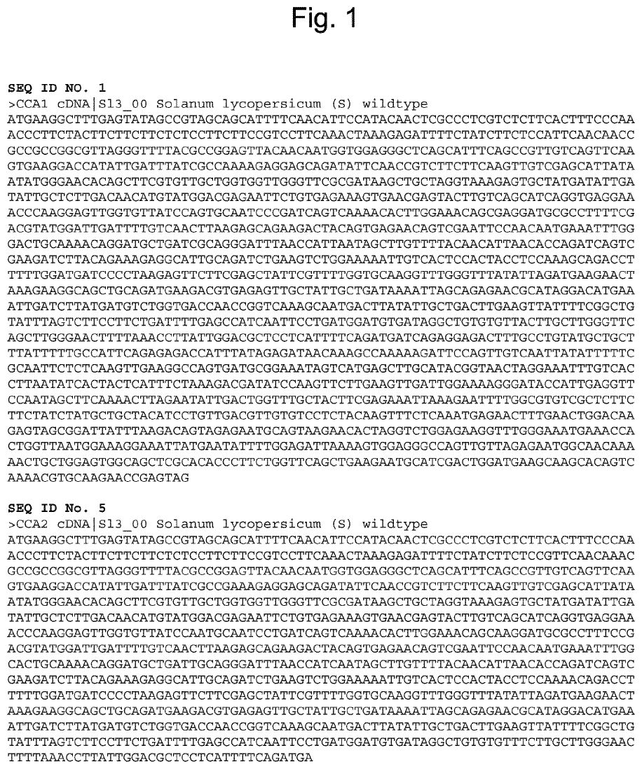

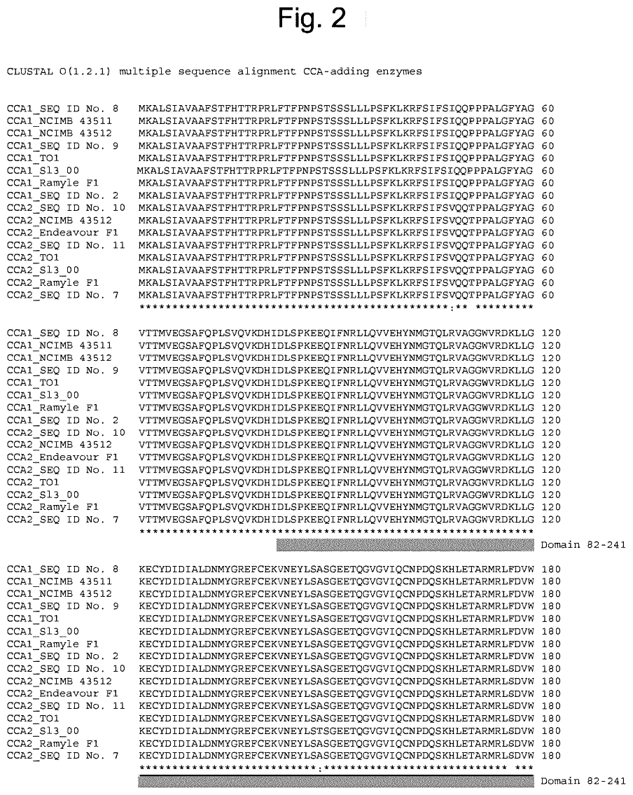

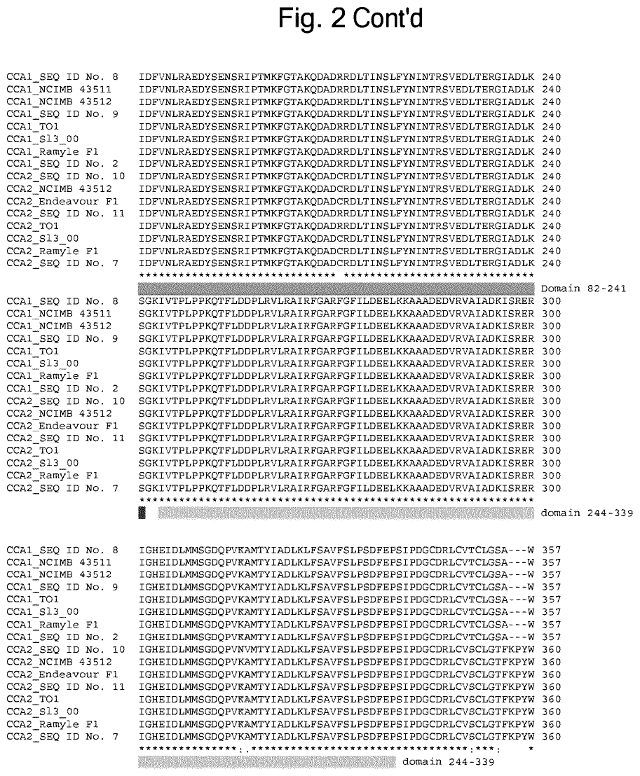

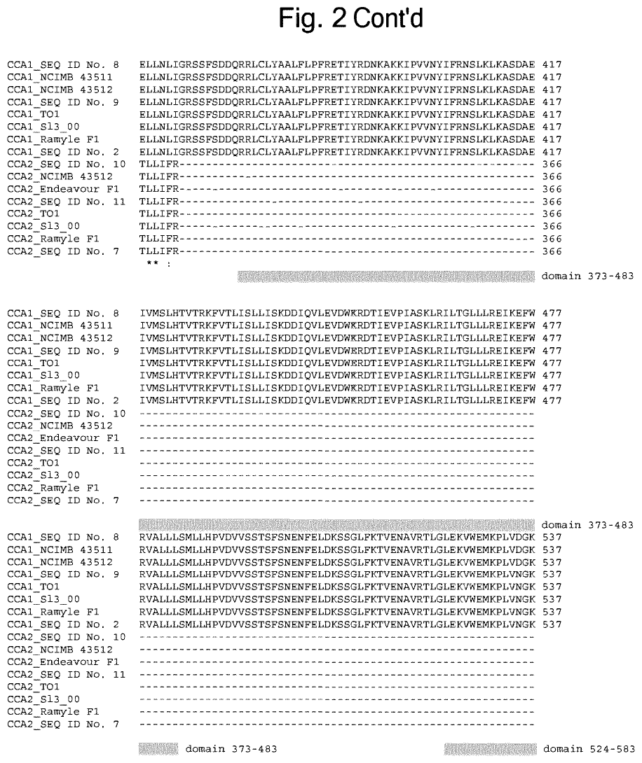

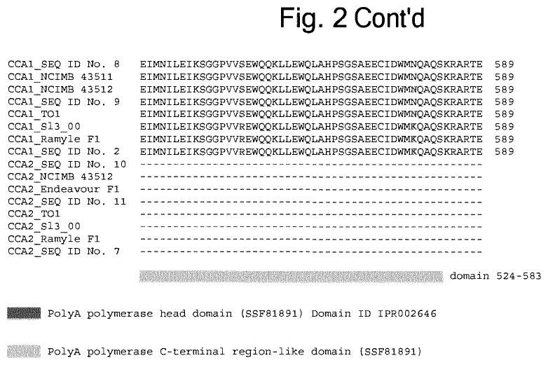

### DETAILED DESCRIPTION OF THE INVENTION

The present invention relates to a modified CCA gene which encodes a CCA-adding enzyme, which modified CCA gene leads to resistance against a positive-strand RNA virus having a TLS, wherein the modified CCA gene is selected from the group consisting of: a gene comprising a nucleotide sequence that encodes a CCA-adding enzyme according to SEQ ID NO: 8, SEQ ID NO: 9, SEQ ID NO: 10, or SEQ ID NO: 11; a gene comprising a promoter sequence comprising SEQ ID NO: 12, SEQ ID NO: 13, SEQ ID NO: 14, SEQ ID NO: 15, or SEQ ID NO: 16; a gene comprising a nucleotide sequence that encodes a CCA-adding enzyme having a deletion, a substitution, or an insertion of at least one amino acid when compared to SEQ ID NO: 2 or SEQ ID NO: 7; a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11; a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; and a gene comprising a promoter sequence having at least 80% sequence identity to SEQ ID NO: 12, SEQ ID NO: 13, SEQ ID NO: 14, SEQ ID NO: 15, or SEQ ID NO: 16. The invention further relates to plants and seeds comprising the modified genes, methods for making and identifying such plants and use of the gene.

The present invention provides a Solanum lycopersicum plant comprising a modified CCA gene on chromosome 11 that leads to Tomato Brown Rugose Fruit Virus (ToBRFV) resistance, wherein the modified CCA gene is selected from the group consisting of:
a gene comprising a nucleotide sequence that encodes a CCA-adding enzyme according to SEQ ID NO: 8, SEQ ID NO: 9, SEQ ID NO: 10, or SEQ ID NO: 11;
a gene comprising a promoter sequence comprising SEQ ID NO: 12, SEQ ID NO: 13, SEQ ID NO: 14, SEQ ID NO: 15, or SEQ ID NO: 16;
a gene comprising a nucleotide sequence that encodes a CCA-adding enzyme having a deletion, a substitution, or an insertion of at least one amino acid when compared to SEQ ID NO: 2 or SEQ ID NO: 7;
a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11;
a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; and
a gene comprising a promoter sequence having at least 80% sequence identity to SEQ ID NO: 12, SEQ ID NO: 13, SEQ ID NO: 14, SEQ ID NO: 15, or SEQ ID NO: 16.

As used herein, a CCA gene is a gene encoding a CCA-adding enzyme. As used herein, a CCA gene is a gene comprising a wildtype CDS sequence represented by SEQ ID NO: 1, or a homologous gene comprising a sequence having at least 80% sequence identity to SEQ ID NO: 1; or a gene encoding a CCA-adding enzyme comprising SEQ ID NO: 2; or a gene encoding a homologous CCA-adding enzyme comprising a sequence having at least 80% sequence identity to SEQ ID NO: 2. As used herein, a gene also comprises the 5′-UTR sequence, the promoter, and the 3′-UTR sequence of that gene.

The promoter of a CCA gene comprises SEQ ID NO: 3, or comprises a sequence having at least 80% sequence identity to SEQ ID NO: 3, preferably 85%, 90%, 93%, 95%, 96%, 97%, 98%, or 99%. A homologous CCA gene comprises a sequence having at least 80% sequence identity to SEQ ID NO: 1, preferably 85%, 90%, 93%, 95%, 96%, 97%, 98%, or 99%. A homologous CCA-adding enzyme comprises a sequence having at least 80% sequence identity to SEQ ID NO: 2, preferably 85%, 90%, 93%, 95%, 96%, 97%, 98%, or 99%.

A CCA-adding enzyme having at least 80% sequence identity to SEQ ID NO: 8 preferably comprises at least one of a N535D substitution, an R553S substitution, or a K579N substitution. A CCA-adding enzyme having at least 80% sequence identity to SEQ ID NO: 9 preferably comprises at least one of a K450E substitution, a R553S substitution, or a K579N substitution. A CCA-adding enzyme having at least 80% sequence identity to SEQ ID NO: 10 preferably comprises at least one of K316N substitution or a A317V substitution. A CCA-adding enzyme having at least 80% sequence identity to SEQ ID NO: 11 preferably comprises at least a C211R substitution. Any of those substitutions is alternatively a substitution on the corresponding position of a homologous sequence.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment.

The CCA-adding enzyme is active in most living organisms, and plays a crucial role therein, as it is essential for adding a CCA-tail to the 3′-end of the universally present transfer RNAs (tRNAs). In nearly all eukaryotes this CCA-tail, which is a prerequisite for aminoacylation of the tRNA, is not encoded by the tRNA gene, and it therefore has to be added post-transcriptionally. The specialized CCA-adding enzyme recognizes all tRNAs and is responsible for synthesis of a proper CCA-tail in all of them. Most eukaryotic genomes have only a single copy of a CCA gene that encodes the essential and highly conserved CCA-adding enzyme.

The CCA-adding enzyme is also involved in other RNA-related processes. One of its tasks is for example tRNA quality control, whereby the enzyme plays a role in tRNA repair, as well as in degradation of unstable or otherwise deviating tRNAs. By adding a double instead of a single CCA tail to RNA that is for some reason identified to be faulty, it tags this RNA for degradation. The CCA-adding enzyme is further also involved in processing of other non-coding RNAs, such as lncRNAs.

Because of the essential role of the CCA-adding enzyme, mutations in a CCA gene, especially mutations that are present in the highly conserved parts of the gene sequence, are anticipated to have a strong negative impact on the growth and development of a plant. Therefore, even though it was known that many viruses, including ToBRFV, have a 3′-terminal transfer RNA-like structure (TLS) that makes use of the CCA-adding enzyme of the host plant for infection of that same host plant, the essential function made CCA genes an unlikely target in an approach to obtain virus resistance.

The present invention however presents a modification in a CCA gene of Solanum lycopersicum that leads to ToBRFV resistance in the plant.

The modification in a CCA gene that leads to resistance against ToBRFV is a modification that is selected from the group consisting of:
a modification in the promoter sequence of a CCA gene;
a modification in the genomic sequence of a CCA gene;
a modification in the coding sequence (CDS) of a CCA gene;
a modification in a regulatory sequence of a CCA gene; and
a modification in a conserved domain of a CCA gene;
or any combination of said modifications.

The modification in a CCA gene that leads to resistance will change the expression of said gene. Alternatively, or as a result, the modification can affect the activity and/or function of the encoded protein, or no protein can be encoded. The modification in the CCA gene of the invention comprises a modification resulting in an amino acid change, a modification resulting in an early stop codon, a modification resulting in a truncated protein, or a modification resulting in a frameshift. Due to the modification the encoded protein has a changed function, a reduced function, or it is non-functional.

The changed expression of the CCA gene of the invention comprises reduced expression, no expression, or silencing. The modification in the CCA gene of the invention comprises a deletion, a substitution, or an insertion of at least one nucleotide in the nucleotide sequence of SEQ ID NO: 1, or in a homologous sequence thereof, or of at least one amino acid in the encoded protein comprising SEQ ID NO: 2, or in a homologous sequence thereof. The modification comprises a modification that affects a conserved domain, such as an active site or catalytic domain, of the encoded protein, which is the CCA-adding enzyme.

In one embodiment, a modification that leads to resistance against a positive-strand RNA virus having a TLS, in particular to ToBRFV, comprises a deletion in the promoter sequence of the CCA gene. The promoter of a CCA gene that is suitable to be modified to result in resistance comprises a sequence having in order of increased preference at least 80%, 85%, 90%, 95%, 98%, 99%, or 100% sequence identity to SEQ ID NO: 3, provided that the promoter sequence comprises SEQ ID NO: 4. A deletion in the promoter sequence of a CCA gene resulting in changed expression of said gene, and thereby in resistance, comprises a deletion in a regulatory sequence, in particular a deletion in the TATA box, or a deletion comprising SEQ ID NO: 4 (Table 1).

In a preferred embodiment, the deletion comprising SEQ ID NO: 4 is a deletion comprising SEQ ID NO: 18, or is a deletion comprising SEQ ID NO: 19.

In one embodiment, the modification that leads to resistance against a positive-strand RNA virus having a TLS comprises a SNP in the CDS of a CCA gene leading to an amino acid substitution. Optionally, the SNP leads to an amino acid substitution in a conserved domain, such as an active site or catalytic domain, of the CCA-adding enzyme. A conserved domain of the CCA-adding enzyme comprises the PolyA_pol_head_domain (domain ID IPR002646, accessible on-line at the InterPro database) comprising positions 82 to 241 of SEQ ID NO: 2, or the corresponding positions in a homologous sequence having at least 80% sequence identity to SEQ ID NO: 2. A conserved domain also comprises the polyA_pol_C-terminal region-like domain (domain ID SSF81891, accessible on-line at the Superfamily database). This domain comprises three active sites and is positioned from amino acid 244 up to amino acid 583 of the CCA-adding enzyme comprising SEQ ID NO: 2, or at the corresponding positions of a homologous CCA-adding enzyme sequence having at least 80% sequence identity to SEQ ID NO: 2.

It was surprisingly found that the species Solanum lycopersicum diverges from the general rule and comprises two CCA genes in its genome, which genes are highly homologous and share 95% sequence identity. The wildtype of the first CCA gene, identified herein as SiCCA1, is represented by SEQ ID NO: 1. The wildtype of the second CCA gene in S. lycopersicum, identified herein as SlCCA2, has an 11 bp deletion when compared to SiCCA1, which deletion results in a frameshift and thereby in an early stop codon. The wildtype of the SlCCA2 gene of S. lycopersicum is represented by SEQ ID NO: 5. The deletion in the SlCCA2 gene as compared to the SiCCA1 gene is present in exon 9 of the gene, and leads to an early stop codon in exon 10 of SlCCA2. The deletion is specifically an 11 bp deletion corresponding to positions 1062 to 1072 of SEQ ID NO: 1. The deletion is in particular a deletion comprising SEQ ID NO: 6 (Table 1). The deletion comprising SEQ ID NO: 6 is present in the wildtype SlCCA2 gene of a susceptible S. lycopersicum plant, and is therefore not a deletion that contributes to ToBRFV resistance.

TABLE 1
Deletions in CCA genes.

| | |
|---|---|
| SEQ ID NO: 4 | ATATTTATTT |
| SEQ ID NO: 6 | TTCAGCTTGGG |
| SEQ ID NO: 18 | TTTTTAAATATTTATTT |
| SEQ ID NO: 19 | AAATATTTATTTTTTTT |

Research has shown that in spite of the early stop codon in the SlCCA2 gene of S. lycopersicum, this gene is still expressed. RNAseq reads spanning the region that has the deletion, such as reads comprising the sequence covering positions 1055 to 1065 of SEQ ID NO: 5, were found. It is therefore expected that the SlCCA2 gene in S. lycopersicum results in a truncated protein. The truncated protein deviates from the SiCCA1 encoded protein after position 350, and terminates after position 366, which is within the polyA_pol_C-terminal region-like domain. As a result, only the first of the three active sites of this domain is still present in the CCA-adding enzyme encoded by SlCCA2. This domain is therefore expected to have a changed, reduced, or no functionality in the enzyme. The CCA-adding enzyme encoded by the wildtype SlCCA2 comprises SEQ ID NO: 7 and has a sequence identity of 90% to SEQ ID NO: 2.

Further research on the wildtype SlCCA2 gene in S. lycopersicum showed that it comprises several polymorphisms when compared to the wildtype SlCCA1, as can be deduced from the sequence alignment of SEQ ID NO: 1 and SEQ ID NO: 5. One of those polymorphisms, a C in SEQ ID NO: 1 versus a T in SEQ ID NO: 5 on position 631, results in an amino acid variant R211C in the wildtype SlCCA2-encoded protein. This position was determined to fall within an essential and highly conserved site of the PolyA_pol_head_domain which is involved in nucleotide binding of the enzyme. Remarkably, it was found that S. lycopersicum lines comprising a mutation that reverts this amino acid substitution in SlCCA2 back from C to R, i.e. a T631C mutation resulting in a C211R substitution as presented in SEQ ID NO: 11, showed a field tolerant ToBRFV phenotype (See also Table 3).

In one embodiment, a modification that leads to resistance against a positive-strand RNA virus having a TLS, in particular to ToBRFV, comprises a T to C SNP on position 631 (T631C) of SEQ ID NO: 5, or on a corresponding position in a homologous sequence thereof, that leads to a C211R amino acid substitution in SEQ ID NO: 7, or on a corresponding position in a homologous sequence thereof. This embodiment particularly relates to genomes that comprise two CCA genes, whereby both CCA genes will have an R on position 211 of the encoded protein after the modification. This embodiment leads to a resistance that comprises at least field tolerance. A plant comprising this modification is preferably a S. lycopersicum plant comprising a modification in the SlCCA2 gene, preferably a modification represented by SEQ ID NO: 11, wherein the modification, i.e. the presence of SEQ ID NO: 11, leads to ToBRFV resistance, in particular to ToBRFV field tolerance.

ToBRFV was first described by Luria et al ((2017): A new Israeli tobamovirus isolate infects tomato plants harboring Tm-2² resistance genes. PLoS ONE 12(1):e0170429. Doi:10.1371/journal.pone.0170429). At the time of that publication Tomato Brown Rugose Fruit Virus was still abbreviated as TBRFV, but in the meantime the commonly used abbreviation for this virus is ToBRFV, which is therefore now also used in the present application.

During even further research, several SlCCA-gene polymorphisms were identified that result in ToBRFV resistance. Certain modifications were found in the CCA genes of wild tomato species, in particular in Solanum pimpinellifolium species; when these modifications were transferred to a ToBRFV susceptible S. lycopersicum plant, the S. lycopersicum plant became resistant to ToBRFV. A SNP resulting in an amino acid change that leads to resistance comprises an A to T SNP on position 948 (A948T) of SEQ ID NO: 1 or SEQ ID NO: 5, a C to T SNP on position 950 (C950T) of SEQ ID NO: 1 or SEQ ID NO: 5, an A to G SNP on position 1348 (A1348G) of SEQ ID NO: 1, an A to G SNP on position 1603 (A1603G) of SEQ ID NO: 1, an A to T SNP on position 1659 (A1659T) of SEQ ID NO: 1, or a G to T SNP on position 1737 (G1737T) of SEQ ID NO: 1, or a SNP on any of the corresponding positions in a homologous sequence of SEQ ID NO: 1. Said nucleotide changes respectively result in a K316N substitution, an A317V substitution, an K450E substitution, an N535D substitution, an R553S substitution, or a K579N substitution in SEQ ID NO: 2, or in an amino acid substitution at the corresponding positions of a homologous sequence of SEQ ID NO: 2. Follow-up research showed that introgression of only the modified CCA1 gene from S. pimpinellifolium to S. lycopersicum already resulted in ToBRFV resistance, in particular of the category tolerance.

As used herein, an X000Y mutation, SNP, or substitution means that the wildtype sequence has a nucleotide or amino acid X on position 000, which is changed to nucleotide or amino acid Y in the modified sequence.

SEQ ID NO: 8 comprises an N535D mutation, an R553S mutation, and a K579N mutation. SEQ ID NO: 9 comprises a K450E, a R553S, and a K579N mutation. SEQ ID NO: 10 comprises a K316N and a A317V mutation.

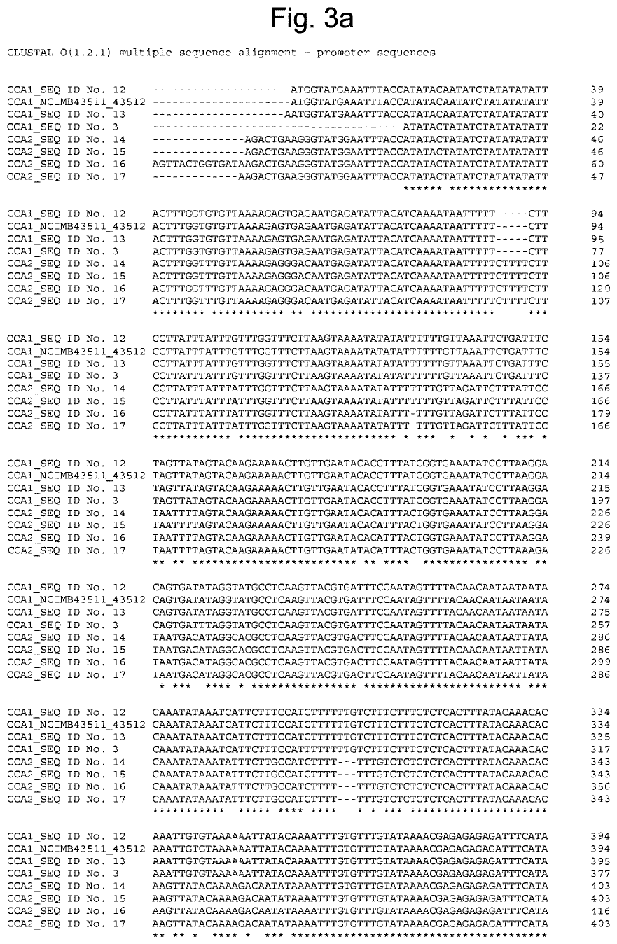

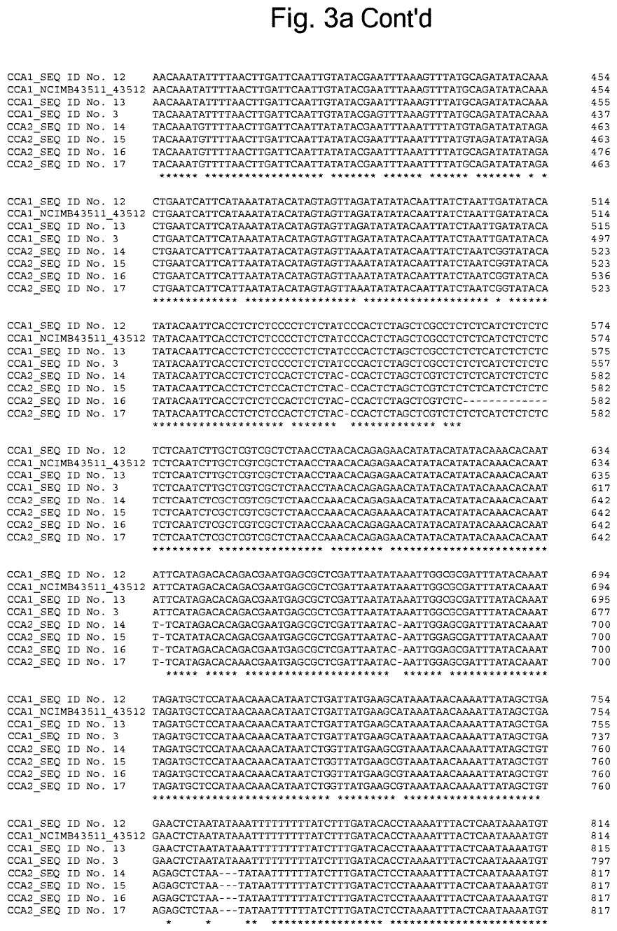

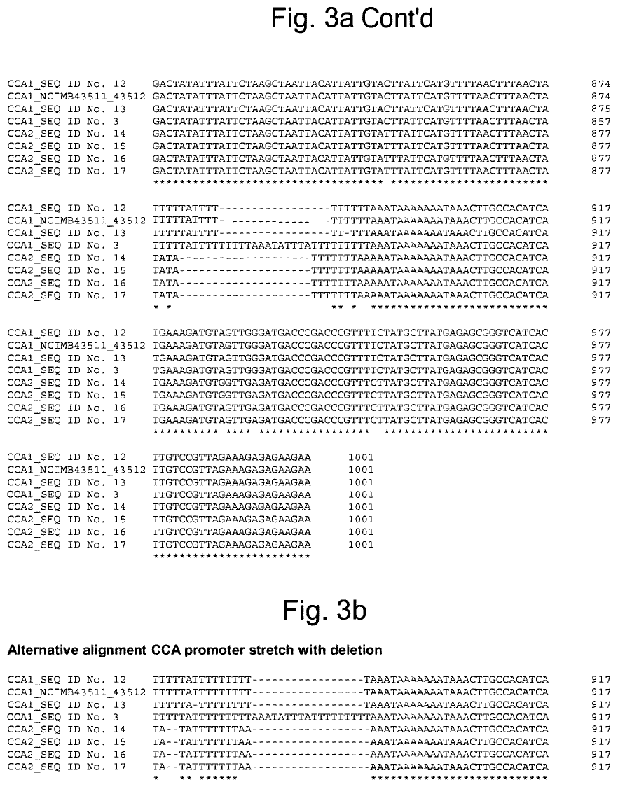

In addition, other polymorphisms that correlated with ToBRFV resistance in S. lycopersicum were found in the promoters of the CCA genes. A CCA1 gene that showed resistance had a deletion comprising SEQ ID NO: 4 in the promoter sequence, when compared to the wildtype promoter sequence comprising SEQ ID NO: 3. Other polymorphisms comprised nucleotide substitutions within the promoter sequence, as for example presented in the promoter sequences alignment of FIG. 3. Remarkably, the wildtype CCA2 gene of S. lycopersicum, comprising SEQ ID NO: 17, also has a deletion comprising SEQ ID NO: 4 when compared to SEQ ID NO: 3. The deletion of SEQ ID NO: 4 appears to be a deletion in the TATA box of the promoter region of the CCA gene. The deletion of SEQ ID NO: 4 therefore is concluded to be related to ToBRFV resistance when it is present in the promoter of a CCA1 gene.

In one embodiment, the promoter of a modified CCA gene of the invention comprises SEQ ID NO: 12, SEQ ID NO: 13, SEQ ID NO: 14, SEQ ID NO: 15, or SEQ ID NO: 16. All of these promoter sequences have a deletion comprising SEQ ID NO: 4 when compared to the wildtype promoter sequence comprising SEQ ID NO: 3 (FIG. 3b).

As used herein, resistance against a positive-strand RNA virus having a TLS, in particular resistance against a Tobamovirus, more in particular resistance against ToBRFV, comprises the categories ‘tolerance’ and/or ‘field tolerance’ to the virus. Virus resistance can express itself on different levels, whereby different mechanisms are involved. When a plant is truly resistant to a virus, the infection and/or replication of the virus in the hostplant is restricted by the resistance mechanism. When a young plant bio-assay is performed, the resistant plant does not show susceptibility symptoms.

As used herein, when a plant is tolerant to a virus, virus replication and multiplication can take place in the plant, which can for example be measured through a qPCR experiment. Some mild symptoms can be observed in a bio-assay, but the impact of the presence of the virus on the fitness of the plant is strongly reduced as compared to the impact on a susceptible plant.

A specific form of tolerance is field tolerance; as used herein, when a plant is field tolerant, the host plant is not able to limit virus replication and multiplication, and the plants will show symptoms in a bio-assay performed under controlled conditions on young plants. However, when such a plant is grown in the field under normal cultivation practices, the host is able to reduce the impact of the virus presence on the plant's fitness, and no or limited symptoms will be seen. In addition, the yield of the crop will not be significantly reduced and will be comparable to the yield of a crop without the virus.

In one embodiment, a modification that leads to resistance against a positive-strand RNA virus having a TLS, in particular to ToBRFV, comprises a combination of two or more of above-described modifications in one CCA gene, which combination can be modifications in the coding sequence, modifications in the promoter sequence, or a modification in the promoter sequence and a modification in the coding sequence. The modification can also be a combination of at least one modification in each of two CCA genes when two CCA genes are present in the genome of a plant, wherein the modifications in both CCA genes can be different or can be the same. The modifications can in particular be a combination of at least one modification in the gene represented by SEQ ID NO: 1, and at least one modification in the gene represented by SEQ ID NO: 5; or a combination of at least one modification in the gene represented by SEQ ID NO: 1 and at least one modification in the promoter represented by SEQ ID NO: 3; or a combination of at least one modification in the gene represented by SEQ ID NO: 5 and at least one modification in the promoter represented by SEQ ID NO: 17, or modifications in homologous sequences of SEQ ID NO: 1, SEQ ID NO: 3, SEQ ID NO: 5, and SEQ ID No 17.

A positive-strand RNA virus having a TLS comprises a virus of the genus Tobamovirus, the genus Tymovirus, or the genus Bromovirus. A positive-strand RNA virus having a TLS is preferably a virus of the genus Tobamovirus, in particular a virus of the species ToBRFV or TMV or ToMV or CGMMV. The modification in the CCA gene of the invention preferably leads to ToBRFV resistance, optionally in combination with resistance against another virus, in particular another Tobamovirus.

The present invention relates to a plant comprising a modified CCA gene of the invention. The plant comprising the modified CCA gene is preferably a plant of the Solanaceae family, comprising a plant of the species Solanum lycopersicum, Capsicum annuum, Solanum melongena, Capsicum frutescens, Solanum tuberosum, Petunia spp, or Nicotiana tabacum. A plant of the invention is preferably a cultivated plant which is non-wild and has agronomical value, and is in particular agronomically elite.

In one embodiment, the plant comprising the modified CCA gene of the invention is resistant to a positive-strand RNA virus having a TLS, in particular to a virus of the genus Tobamovirus, the genus Tymovirus, or the genus Bromovirus, preferably a virus of the genus Tobamovirus. The positive-strand RNA virus is most preferably the species Tomato Brown Rugose Fruit Virus (ToBRFV), or the species Tobacco Mosaic Virus (TMV), or the species Tomato Mosaic Virus (ToMV).

In a preferred embodiment, the plant of the invention is a plant of the species Solanum lycopersicum comprising a modified CCA gene, which plant is resistant to a Tobamovirus, in particular to Tomato Brown Rugose Fruit Virus (ToBRFV). The modification in a CCA gene in the S. lycopersicum plant of the invention comprises a modification of an A to T SNP on position 948 of SEQ ID NO: 1 and/or SEQ ID NO: 5; a C to T SNP on position 950 of SEQ ID NO: 1 and/or SEQ ID NO: 5; an A to G SNP on position 1348 of SEQ ID NO: 1; an A to G SNP on position 1603 of SEQ ID NO: 1; an A to T SNP on position 1659 of SEQ ID NO: 1; a G to T SNP on position 1737 of SEQ ID NO: 1; a T to C SNP on position 631 of SEQ ID NO: 5; a deletion in the promoter of the CCA gene, in particular a deletion comprising SEQ ID NO: 4 from the promoter sequence comprising SEQ ID NO: 3; or corresponding modifications in homologous sequences of SEQ ID NO: 1, SEQ ID NO: 3 and SEQ ID NO: 5. A certain modification in a CCA gene can result in resistance of one or more categories of resistance.

An overview of SNP modifications in a CCA gene resulting in amino acid substitutions in its encoded protein, which is the CCA-adding enzyme, that form part of the present invention is presented in Table 4. The modification is indicated from susceptible (before the specified position) to resistant (after the specified position). In a preferred embodiment a plant of the invention comprises one or more or all of the modifications that are presented herein for the modified CCA1 gene, which modifications comprises the SNPs and deletions as presented herein.

In one embodiment, a S. lycopersicum plant of the invention comprises a modified CCA1 gene. In one embodiment, a S. lycopersicum plant of the invention comprises a CCA1 gene comprising SEQ ID NO: 8 and SEQ ID NO: 12; or the plant comprises a CCA1 gene comprising SEQ ID NO: 9 and SEQ ID NO: 13. In one embodiment, a S. lycopersicum plant of the invention comprises two modified CCA genes. In one embodiment, a S. lycopersicum plant of the invention comprises a CCA1 gene comprising SEQ ID NO: 8 and SEQ ID NO: 12 and a CCA2 gene comprising SEQ ID NO: 10 and SEQ ID NO: 14; or the plant comprises a CCA1 gene comprising SEQ ID NO: 8 and SEQ ID NO: 12 and a CCA2 gene comprising SEQ ID NO: 7 and SEQ ID NO: 15; or the plant comprises a CCA1 gene comprising SEQ ID NO: 9 and SEQ ID NO: 13 and a CCA2 gene comprising SEQ ID NO: 11 and SEQ ID NO: 16.

ToBRFV resistance is determined by comparison to a control variety known to be ToBRFV susceptible (S). Examples of ToBRFV susceptible tomato varieties that can be used as controls are Endeavour F1 and Ramyle F1. As a resistant control a plant deposited as NCIMB 42882, or NCIMB 42885, or NCIMB 42887, or NCIMB 42890, or NCIMB 43511, or NCIMB 43512 can be used; a plant of these deposits comprises a modified CCA gene of the invention. NCIMB 42882 and NCIMB 43511 comprise a CCA1 gene encoding SEQ ID NO: 8 and a CCA2 gene encoding SEQ ID NO: 10. NCIMB 42885, NCIMB 42887, NCIMB 42890, and NCIMB 43512 comprise a CCA1 gene encoding SEQ ID NO: 8 and a CCA2 gene encoding SEQ ID NO: 7. The promoter of the CCA1 gene of NCIMB 42882, NCIMB 42885, NCIMB 42887, NCIMB 42890, NCIMB 43511, and NCIMB 43512 comprises SEQ ID No 12. The promoter of the CCA2 gene of NCIMB 42882 and NCIMB 43511 comprises SEQ ID NO: 14. The promoter of the CCA2 gene of NCIMB 42885, NCIMB 42887, NCIMB 42890, and NCIMB 43512 comprises SEQ ID NO: 15.

To determine resistance, seeds of the accessions to be tested are sown in standard seedling trays and seedlings are inoculated 4 weeks after sowing. Inoculum is prepared by grounding leaves of tomato plants that were infected with ToBRFV in a 0.01 M phosphate buffer (pH 7.0) mixed with celite. The seedlings are then dusted with carborundum powder prior to gently rubbing the leaf with inoculum. Resistance is suitably scored on a scale of 0-5; the description of the scales of the scores can be found in Table 2. Observation of the symptoms on the young tomato plants in the bio-assay is preferably done 14-21 days after inoculation (dai).

As used herein, a Solanum lycopersicum plant that is resistant to ToBRFV due to the presence of a modified CCA gene has a score that is 3 or lower than 3, preferably lower than 2.5, when scoring according to Table 2 is used and a bio-assay as described above is performed. In one embodiment, a plant is tolerant to ToBRFV and has a score of 2 or lower than 2, preferably a score of 1 or lower than 1. In another embodiment a plant has field tolerance (FT) to ToBRFV, and has a score of 3 or lower than 3, preferably lower than 2.5, in a bio-assay, but has a score of 2 or lower than 2 in field conditions. As is a criterion in any bio-assay, a representative number of plants has to be scored to obtain a reliable rating, for example 10 plants of a certain line, and the average score should be taken. The susceptible (S) controls in this test should have a score that is higher than 3, preferably higher than 3.5, when the test is performed properly.

A plant of the invention comprises a modified CCA gene homozygously or heterozygously, i.e. a modified CCA gene can be present on both chromosomes of a chromosome pair in the genome of a plant, or on only one chromosome of a chromosome pair. When two modified CCA genes are present in a certain species, for example in Solanum lycopersicum, they can be present in coupling phase, i.e. two modified CCA genes on the same chromosome, or in repulsion phase, i.e. one modified CCA gene on each complementary chromosome. A plant of the invention comprises a plant of an inbred line, a hybrid, an open pollinated variety, a doubled haploid, or a plant of a segregating population.

In one embodiment, a plant of the invention is a Solanum lycopersicum plant comprising a modified CCA gene as comprised in the genome of a S. lycopersicum plant representative seed of which was deposited with the NCIMB under deposit number NCIMB 42882, or NCIMB 42885, or NCIMB 42887, or NCIMB 42890, NCIMB 43511, or NCIMB 43512.

In one embodiment, a plant of the invention is a Solanum lycopersicum plant deposited as NCIMB 42882, or NCIMB 42885, or NCIMB 42887, or NCIMB 42890, NCIMB 43511, or NCIMB 43512, or a progeny plant thereof comprising one or more or all polymorphisms in the CCA genes that are present in said deposits.

The virus resistance, in particular the ToBRFV resistance, in a plant of the present invention inherits in an intermediate manner. As used herein, intermediate means that a higher level of resistance is found when a modified CCA gene of the invention is homozygously present. The heterozygous presence of a modified CCA gene of the invention however still confers a certain level of ToBRFV resistance. The heterozygous level is especially displayed when virus pressure is not too high. The ToBRFV resistance of both homozygous and heterozygous plants makes the plants more suitable for cultivation under conditions where ToBRFV is present. The improvement on a heterozygous level can also be expressed when the heterozygous plant has two different modified CCA genes, whereby each modified CCA gene comes from a different parent. Therefore both heterozygous and homozygous plants are considered to have improved agronomic characteristics. In addition, heterozygous plants can be used as sources for development of homozygous plants through crossing and selection, which heterozygous plants also for that reason form a part of this invention.

The invention further relates to a seed that comprises a modified CCA gene of the invention, which seed can grow into a plant of the invention. The invention also relates to use of said seed for the production of a plant of the invention, by growing said seed into a plant. The seed is preferably a seed of S. lycopersicum. The seed preferably comprises the modified CCA gene homozygously. The invention also relates to a plant part of a plant of the invention, comprising a fruit of a plant of the invention or a seed of a plant of the invention, wherein the plant part comprises a modified CCA gene in its genome. The plant part is preferably a part of a S. lycopersicum plant.

The invention further relates to a method for seed production comprising growing a plant from a seed of the invention, allowing the plant to produce a fruit with seed, harvesting the fruit, and extracting those seed. Production of the seed is suitably done by selfing or by crossing with another plant that is optionally also a plant of the invention. The seed that is so produced has the capability to grow into a plant that is resistant to a positive-strand RNA virus having a TLS, in particular to a virus of the genus Tobamovirus, and more in particular to ToBRFV.

The invention further relates to hybrid seed and to a method for producing said hybrid seed, comprising crossing a first parent plant with a second parent plant and harvesting the resultant hybrid seed, wherein the first parent plant and/or the second parent plant is a plant of the invention comprising a modified CCA gene of the invention. In a preferred embodiment both parent plants comprise a modified CCA gene of the invention. The resulting hybrid plant that can be grown from the hybrid seed, comprising the modified CCA gene of the invention, which hybrid plant has resistance to a positive-strand RNA virus having a TLS, in particular to a virus of the genus Tobamovirus, and more in particular to ToBRFV, is also a plant of the invention. The resulting hybrid plant is preferably homozygous for the modified CCA gene of the invention.

The present invention relates to a method for producing a plant that is resistant to a positive-strand RNA virus having a TLS, in particular to a virus of the genus Tobamovirus, and more in particular to ToBRFV, comprising introducing a modification in a CCA gene, which modification leads to resistance. Said method comprises the introduction of a deletion, a substitution, or an insertion in the coding sequence and/or the promoter sequence of a CCA gene. The introduction of such a modification can be done by a mutagenesis approach using a chemical compound, such as ethyl methane sulphonate (EMS); or by using physical means, such as UV-irradiation, fast neutron exposure, or other irradiation techniques. In one embodiment the mutation that is introduced comprises the introduction of an A to T SNP on position 948 (A948T) of SEQ ID NO: 1 or SEQ ID NO: 5, a C to T SNP on position 950 (C950T) of SEQ ID NO: 1 or SEQ ID NO: 5, an A to G SNP on position 1348 (A1348G) of SEQ ID NO: 1, an A to G SNP on position 1603 (A1603G) of SEQ ID NO: 1, an A to T SNP on position 1659 (A1659T) of SEQ ID NO: 1, or a G to T SNP on position 1737 (G1737T) of SEQ ID NO: 1, or on any of the corresponding positions in a homologous sequence of SEQ ID NO: 1. In one embodiment the introduced mutation comprises a deletion in the promoter of the CCA gene, in particular a deletion comprising SEQ ID NO: 4 in the promoter sequence comprising SEQ ID NO: 3, or a deletion comprising SEQ ID NO: 4 in a homologous promoter sequence of SEQ ID NO: 3. In one embodiment a combination of above-defined modifications is introduced in a plant.

Introduction of a modification can also be done using a more specific, targeted approach including targeted genome editing by means of homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

Introduction of a modified CCA gene of the invention can also be done through introgression from a plant comprising said modified CCA gene, for example from a plant that was deposited as NCIMB 42882, or NCIMB 42885, or NCIMB 42887, or NCIMB 42890, or NCIMB 43511, or NCIMB 43512, or from progeny thereof, or from another plant that is resistant to a positive-strand RNA virus having a TLS, in particular to a virus of the genus Tobamovirus, and more in particular to ToBRFV, and in which a modified CCA gene was identified. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied. Progeny of a deposit can be sexual or vegetative descendants of that deposit, which can be selfed and/or crossed, and can be of an F1, F2, or further generation, as long as the descendants of the deposit still comprise a modified CCA gene of that deposit. A plant produced by such method is also a part of the invention.

In one embodiment a modified CCA gene is introgressed into S. lycopersicum from a plant of the species S. pimpinellifolium. In another embodiment a modified CCA gene is introgressed from a S. lycopersicum plant comprising the modified CCA gene into a S. lycopersicum plant lacking a modified CCA gene, or into a S. lycopersicum plant comprising a different modification in an, optionally different, CCA gene. In a preferred embodiment at least a modified CCA1 gene is introgressed.

Transgenic techniques used for transferring sequences between plants that are sexually incompatible can also be used to produce a plant of the invention, by transferring a modified CCA gene from one species to another. Techniques that can suitably be used comprise general plant transformation techniques known to the skilled person, such as the use of an Agrobacterium-mediated transformation method.

The invention also relates to a method for the production of a plant which is resistant to a positive-strand RNA virus having a TLS, in particular to ToBRFV, said method comprising:
crossing a plant of the invention comprising a modified CCA gene of the invention with another plant;
optionally performing one or more rounds of selfing and/or crossing a plant resulting from step a) to obtain a further generation population;
selecting from the population resulting from the cross of step a), or from the further generation population of step b), a plant that comprises a modified CCA gene as defined herein, which plant is resistant against a positive-strand RNA virus having a TLS, in particular to ToBRFV.

Selection of a plant comprising a modified CCA gene comprises identification of any of the modifications as described herein, in particular the SNP modifications as presented in Table 4, suitably by using a marker that is designed to identify such modification. The sequence of a marker designed to identify such modification comprises that particular modification as well as an appropriate number of nucleotides on both sides of the modification, for example at least 5, 10, 15, or 20 nucleotides on each side.

The invention also relates to a method for the production of a plant which is resistant to a positive-strand RNA virus having a TLS, in particular to ToBRFV, said method comprising:
crossing a first parent plant of the invention comprising a modified CCA gene of the invention with a second parent plant, which is another plant does not comprise a modified CCA gene of the invention, or is another plant that comprises a different modification in a CCA gene;
backcrossing the plant resulting from step a) with the second parent plant for at least three generations;
selecting from the third or higher backcross population a plant that comprises at least the modified CCA gene of the first parent plant of step a).

The invention additionally provides for a method of introducing another desired trait into a plant that is resistant to a positive-strand RNA virus having a TLS, in particular to ToBRFV, comprising:
crossing a plant comprising a modified CCA gene of the invention with a second plant that comprises the other desired trait to produce F1 progeny;
optionally selecting in the F1 for a plant that comprises the virus resistance and the other desired trait;
crossing the optionally selected F1 progeny with one of the parents for at least three generations, to produce backcross progeny;
selecting backcross progeny comprising the virus resistance and the other desired trait; and
optionally repeating steps c) and d) one or more times in succession to produce selected fourth or higher backcross progeny that comprises the virus resistance and the other desired trait.

Optionally, selfing steps are performed after any of the crossing or backcrossing steps. Selection of a plant comprising virus resistance and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and a fruit obtained therefrom.

The invention further relates to a method for the production of a plant comprising a modified CCA gene of the invention, which plant is resistant to a positive-strand RNA virus having a TLS, in particular to a Tobamovirus, and more specifically to ToBRFV, by using tissue culture or by using vegetative propagation.

The present invention relates to a method for identification of a plant comprising a modified CCA gene of the invention, which plant is resistant to a positive-strand RNA virus having a TLS, in particular to ToBRFV, wherein the identification comprises determining the presence of a modification in the CCA gene of SEQ ID NO: 1, or in a homologous sequence thereof, and analyzing if the plant comprising the modification is resistant to a positive-strand RNA virus having a TLS, in particular to a Tobamovirus, and more specifically to ToBRFV. Determining the presence of a modification in a CCA gene comprises identification of any of the modifications as described herein, in particular the SNP modifications as presented in Table 4, suitably by using a marker that is designed to identify such modification as its sequence comprises that particular modification as well as an appropriate number of nucleotides on both sides of the modification, for example at least 5, 10, 15, or 20 nucleotides on each side.

The present invention further relates to a method of selection of a plant which is resistant to a positive-strand RNA virus having a TLS, in particular to ToBRFV, the method comprising identification of a modified CCA gene of the invention in a plant, preferably by using a marker, and subsequently selecting said plant as a plant which is resistant to a positive-strand RNA virus having a TLS, in particular to a Tobamovirus, and more specifically to ToBRFV. Optionally the virus resistance can be confirmed by performing a bio-assay as described in Example 1. The selected plant obtained by such method is also a part of this invention.

The invention also relates to propagation material suitable for producing a plant of the invention, wherein the propagation material is suitable for sexual reproduction, and is in particular selected from a microspore, pollen, an ovary, an ovule, an embryo sac, or an egg cell; or the propagation material is suitable for vegetative reproduction, and is in particular selected from a cutting, a root, a stem cell, or a protoplast; or the propagation material is suitable for tissue culture of regenerable cells, and is in particular selected from a leaf, pollen, an embryo, a cotyledon, a hypocotyl, a meristematic cell, a root, a root tip, an anther, a flower, a seed and a stem. A plant produced from the propagation material comprises the modified CCA gene of the invention that provides resistance to a positive-strand RNA virus having a TLS, in particular to ToBRFV. A plant of the invention may be used as a source of the propagation material. A tissue culture comprising regenerable cells also forms a part of this invention.

The invention further relates to a cell of a plant of the invention as defined herein. A cell of the invention can be obtained from, or be present in, a plant of the invention. Such a cell may either be in isolated form, or a part of a complete plant, or parts thereof, and still forms a cell of the invention because such a cell comprises the modified CCA gene of the invention. Each cell of a plant of the invention carries the modified CCA gene of the invention. A cell of the invention may also be a regenerable cell that can regenerate into a new plant of the invention.

The invention further relates to plant tissue of a plant of the invention, comprising the modified CCA gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention moreover relates to progeny of a plant, a cell, a tissue, or a seed of the invention, which progeny comprises the modified CCA gene of the invention. Such progeny can in itself be a plant, a cell, a tissue, or a seed. The progeny can in particular be progeny of a plant of the invention deposited under NCIMB number NCIMB 42882, or NCIMB 42885, or NCIMB 42887, or NCIMB 42890, or NCIMB 43511, or NCIMB 43512. As used herein, progeny comprises the first and all further descendants from a cross with a plant of the invention, wherein a cross comprises a cross with itself or a cross with another plant, and wherein a descendant that is determined to be progeny comprises the modified CCA gene of the invention. The modified CCA gene of the invention can be present in homozygous or heterozygous form in the progeny. In one embodiment descendants can be obtained through selfing and/or further crossing of any of the deposited seeds described herein. Progeny also encompasses plant material comprising the modified CCA gene of the invention that is obtained by vegetative propagation, or another form of multiplication, from a plant of the invention.

The invention also relates to a marker for the identification of a modified CCA gene in a plant, which marker comprises any of the modifications in a CCA gene as described herein and can thereby identify said modifications. A marker of the invention is in particular a marker comprising, and thereby suitable for identifying, a SNP modification, i.e. a polymorphism, as presented in Table 4, comprising the modifications of an A to T SNP on position 948 of SEQ ID NO: 1; a C to T SNP on position 950 of SEQ ID NO: 1; an A to G SNP on position 1348 of SEQ ID NO: 1; an A to G SNP on position 1603 of SEQ ID NO: 1; an A to T SNP on position 1659 of SEQ ID NO: 1; a G to T SNP on position 1737 of SEQ ID NO: 1; a T to C SNP on position 631 of SEQ ID NO: 5; or for identifying a deletion in SEQ ID NO: 3 comprising SEQ ID NO: 4; or wherein the marker detects a modification on a corresponding position of a homologous sequence having at least 80% sequence identity to SEQ ID NO: 1 or SEQ ID NO: 3 or SEQ ID NO: 5. The marker sequence to be used for identification of the SNP or deletion comprises at least 5, 10, 15, or 20 nucleotides on each side of the position of the SNP or deletion, which nucleotides are as present in SEQ ID NO: 1 or SEQ ID NO: 3 or SEQ ID NO: 5. The use of such marker for identification of a modified CCA gene in a plant, in particular in a plant of the species Solanum pimpinellifolium or Solanum lycopersicum, is also part of this invention.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Bio-Assay for ToBRFV Resistance in Solanum lycopersicum

S. lycopersicum lines that were derived from S. pimpinellifolium sources with ToBRFV resistance, and having modifications in one or both CCA genes, were observed in a ToBRFV bio-assay. As resistant controls three S. pimpinellifolium sources were included. As susceptible controls Endeavour F1 and Ramyle F1 were used.

Seeds of the accessions to be tested were sown in standard seedling trays and 10 seedlings per accession were inoculated 4 weeks after sowing. Inoculum was prepared by grounding leaves of tomato plants that were infected with ToBRFV in a 0.01 M phosphate buffer (pH 7.0) mixed with celite. Plants were dusted with carborundum powder prior to gently rubbing the leaf with inoculum. Scoring of the symptoms was done according to Table 2 at 19 days after inoculation.

TABLE 2
scoring scales ToBRFV resistance

| score | Symptoms |
|---|---|
| 0 | No symptoms |
| 1 | Not clean, a single spot, some minor discoloration |
| 2 | Mosaic, clear visible symptoms |
| 3 | Severe mosaic, starting deformation in the head |
| 4 | Severe mosaic, necrosis on the stem, serious |
| | deformation in the head, spots in blisters |
| 5 | Dead plant |

Results of the bio-assay are presented in Table 3; the average score of the 10 inoculated seedlings is given. The resistant controls all showed a score of 0.5 or lower, while the susceptible controls were heavily infected and scored 4.0 or higher—quite a number of those were dead at the moment of scoring. T0310 and T0311 are S. lycopersicum lines of a BC3F4 and a BC4F3 generation, respectively, that are homozygous for the CCA genes derived from GNL.3919. T0313 is a cross between a line with the CCA genotype of NCIMB 43511 and a line with the CCA genotype of NCIMB 43512, i.e. a cross between S. lycopersicum lines derived from sources GNL.3951 and GNL.3919. T0314 is a BC4F3 S. lycopersicum line that is homozygous for the CCA genes derived from GNL.3951. T0315 is an F1 between two S. lycopersicum lines that were both homozygous for the CCA genes derived from GNL.3951. A ‘CCA genotype’ means the line has the sequence of CCA1 and CCA2 as in the referred deposit, which genotypes are presented in Table 4. The ‘CCA genotype’ of NCIMB 43511 is derived from GNL.3951, and is also the genotype of NCIMB 42882. The ‘CCA genotype’ of NCIMB 43512 is derived from GNL.3919, and is also the genotype of NCIMB 42885, NCIMB 42887, and NCIMB 42890. NCIMB 42882, NCIMB 42885, NCIMB 42887, and NCIMB 42890 are S. lycopersicum F3 lines having the CCA genes of the indicated sources homozygously.

All S. lycopersicum lines in which the CCA genes from the S. pimpinellifolium sources were introgressed showed a very convincing level of ToBRFV resistance, and are considered to fall in the resistance category ‘tolerant’, as symptoms stayed well below a score of 2.0. Another line, TO1, which comprised a different modification, had a score lower than 3.0; however, in later field assays it performed nicely and was clearly better than susceptible controls (results not shown). A line having this modification was determined to fall in the resistance category ‘field tolerant’.

TABLE 3
ToBRFV bio-assay scores

| | bio-assay | |
|---|---|---|
| Accession | score | |
| GNL.3951 | 0.3 | resistant S. pimpinellifolium control |
| GNL.3919 | 0.5 | resistant S. pimpinellifolium control |
| Ramyle F1 | 4.0 | susceptible control |
| Endeavour F1 | 5.0 | susceptible control |
| TO1 | 2.8 | field tolerant line comprising C211R in CCA2 |
| TO310 | 1.7 | CCA genotype of NCIMB 43512 |
| TO311 | 1.3 | CCA genotype of NCIMB 43512 |
| TO313 | 1.5 | CCA genotype of NCIMB 43511 and 43512 |
| TO314 | 1.2 | CCA genotype of NCIMB 43511 |
| TO315 | 1.2 | CCA genotype of NCIMB 43511 |

### Example 2: QTL Mapping and Marker Development

In order to map ToBRFV resistance conferring QTLs from identified S. pimpinellifolium sources, 184 plants of F2 populations from crosses between S. pimpinellifolium sources and S. lycopersicum were phenotyped for ToBRFV resistance; parents were also included for reference; DNA samples were taken of each plant for genotyping. Phenotypic scores 0 to 4 according to Table 2 were present in all F2 populations.

Per population a genetic map was constructed; non-polymorphic markers and markers with a strong segregation distortion were removed. For each population around 400 to 450 markers were mapped that were well-distributed over the genome with an average spacing of 2-3 cM. The marker order was determined; the public genome assembly was used to determine numbering and orientation of the linkage groups. From the seven individual maps a consensus map was created.

Phenotypic scores, genotypic data, and a consensus map containing marker positions were used as input data for the QTL mapping. QTL analysis was performed, and mapping of the data initially resulted in the identification of three QTLs: one on chromosome 11, one on chromosome 12, and one on chromosome 6.

Through further observations on populations that segregated for resistance, and subsequent fine-mapping of the QTLs, it was found that the main contribution to resistance to TBRFV was due to the presence of the QTL on chromosome 11. Through this fine-mapping the QTL region on chromosome 11 could be further narrowed down. Identification of recombinants within the original QTL initially resulted in a smaller region of approximately 54 Mbp. Even further fine-mapping resulted in a small region comprising an introgression from the source between around 7.7 Mbp and 10.1 Mbp. The final recombinants that showed resistance only had a small introgression from the source between around 8.6 and 10.1 Mbp.

### Example 3: Identification of Modifications in CCA Genes that Lead to ToBRFV Resistance

Various Solanum lycopersicum populations, derived from crosses that segregated for ToBRFV resistance as described in Example 2, were further fine mapped to a small region on chromosome 11 that contained only four potential genes which were likely to contribute to the ToBRFV resistance. Whole genome sequences were available in-house for the backgrounds of the resistant and susceptible lines that were used in the development of these populations. Therefore, a SNP-calling approach for the region was done, which means unique polymorphisms in the region were identified through comparing the sequences to each other.

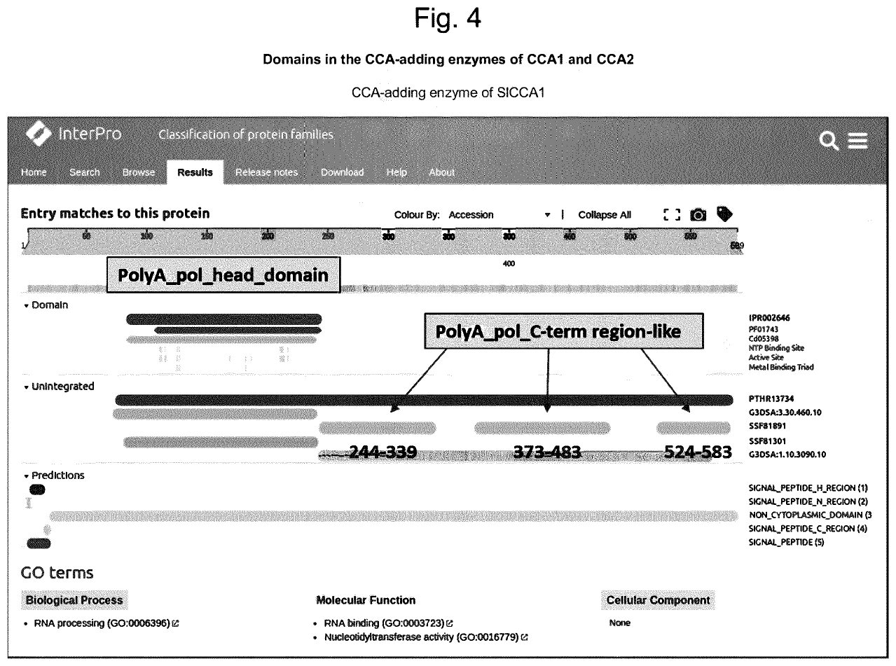

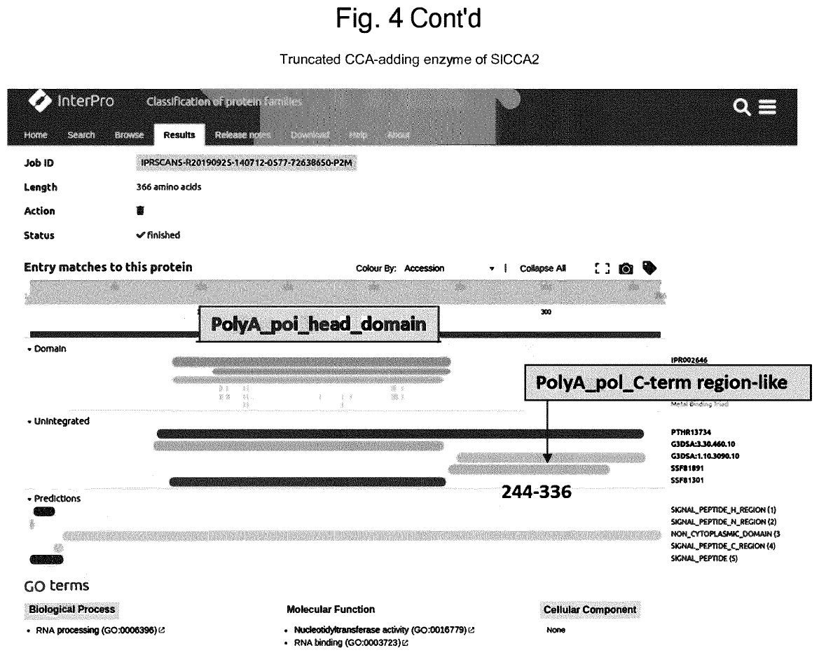

Among the genes in the region of interest were two CCA genes, which were designated CCA1 and CCA2. CCA1 was found to be a complete CCA gene, which had various polymorphisms between susceptible and resistant material, but all of them led to a protein that harbored the essential domains and active sites of a CCA-adding enzyme. The CCA2 gene however also contained various polymorphisms, but in all lines, including the susceptible material, the CCA2 gene had an early stop codon which resulted in a truncated protein that did no longer contain all essential active sites of a CCA-adding enzyme. The encoded protein was truncated within the polyA_pol_C-terminal region-like domain, and as a result only the first of three active sites of this domain is still present in the CCA2 gene of S. lycopersicum (FIG. 4).

Different resistant lines were observed to have different polymorphisms. A number of the polymorphisms resulted in non-conservative amino acid changes, which polymorphisms are presented in Table 4.

TABLE 4
Certain SNP modifications correlating with ToBRFV resistance in S. lycopersicum

| | Gene in S. lycopersicum | | | | | | |
|---|---|---|---|---|---|---|---|
| | | CCA2 | CCA2 | | | | |
| | CCA2 | or CCA1 | or CCA1 | CCA1 | CCA1 | CCA1 | CCA1 |
| CDS | T631C | A948T | C950T | A1348G | A1603G | A1659T | G1737T |
| protein | C211R | K316N | A317V | K450E | N535D | R553S | K579N |
| CCA polymorphisms | C | T | T | G | G | T | T |
| correlating with resistance | | | | | | | |
| Polymorphisms present | T | T | T | A | G | T | T |
| in NCIMB 43511 | | | | | | | |
| Polymorphisms present | T | A | C | A | G | T | T |
| in NCIMB 43512 | | | | | | | |
| Wildtype v3 public genome | T | A | C | A | A | A | G |
| S. lycopersicum (Sl3_00) | | | | | | | |

In addition, it was found that the presence of ToBRFV resistance correlated with a deletion in the promoter of the CCA1 gene. In all situations wherein there was a deletion in the promoter, this deletion comprised at least the sequence ATATTTATTT (SEQ ID NO: 4; Table 1), but the deletion could also have several nucleotides more, for example one to ten nucleotides more, in addition to just a deletion of SEQ ID NO: 4. In a certain case it was for example found that the deletion comprised the sequence represented by SEQ ID NO: 18, or the sequence represented by SEQ ID NO: 19, both of which comprise SEQ ID NO: 4. The SEQ ID NO: 4 stretch within SEQ ID NOS: 18 and 19 is underlined in Table 1.

The deletion in the promoter was present in a TATA rich region, and is therefore believed to be a deletion in the TATA-box of the promoter.

Through analysis of the correlation in segregation of phenotypes and genotypes it was determined that a modification in a CCA gene, which can be a modification in the CCA1 gene and/or a modification in the CCA2 gene, and which can be a modification in the promoter and/or in the coding sequence, was the cause of the ToBRFV resistance of the resistant Solanum lycopersicum plants. Further analysis of S. lycopersicum plants, that had a recombination between the CCA1 and CCA2 gene derived from the S. pimpinellifolium sources, showed that the presence of the modified CCA1 gene was sufficient to obtain the ToBRFV resistance level described herein.

### Example 4: Modification of a CCA Gene to Obtain Resistance to a Positive-Strand RNA Virus Having a TLS

Modifications are introduced in seed of a plant of interest in which resistance to a positive-strand RNA virus having a TLS is needed, for example resistance to a Tobamovirus, such as ToBRFV, ToMV, or TMV. The modification is introduced through mutagenesis, such as an EMS treatment, through radiation means, or through a specific targeted approach, such as CRISPR. When a non-targeted approach such as EMS is used, this is combined with an identification technique such as TILLING. In this way, both for mutagenesis as well as a targeted modification means, a modification in a CCA gene can be generated and identified. The skilled person is familiar with these means for introducing modifications into the genome of a plant of interest.

Modified seed is then germinated and plants are grown, which are crossed or selfed to generate M2 seed. Subsequently a plant screen is performed to identify the modifications in a CCA gene, based on comparison to the wildtype sequence of the one or more CCA genes of that species. For Solanum lycopersicum for example, comparison to SEQ ID NO: 1, SEQ ID NO: 3, SEQ ID NO: 5, or SEQ ID NO: 17 can be done. The skilled person is familiar with TILLING to identify mutations in specific genes (McCallum et. al. (2000) Nature Biotechnology, 18: 455-457), and with techniques for identifying nucleotide changes such as DNA sequencing, amongst others.

Plants with a modified CCA gene are homozygous or made homozygous by selfing, crossing, or the use of doubled haploid techniques which are familiar to the skilled person. Plants identified and selected on the basis of a modification in a CCA gene can then be tested for resistance to a positive-strand RNA virus having a TLS, for example resistance to a Tobamovirus, such as ToBRFV, ToMV, or TMV. A plant that is produced, identified and selected in this way is confirmed to have their virus resistance as a result from one or more modifications in the CCA gene.

The invention is further described by the following numbered paragraphs:

1. A Solanum lycopersicum plant comprising a modified CCA gene on chromosome 11 that leads to Tomato Brown Rugose Fruit Virus (ToBRFV) resistance, wherein the modified CCA gene is selected from the group consisting of:
a gene comprising a nucleotide sequence that encodes a CCA-adding enzyme according to SEQ ID NO: 8, SEQ ID NO: 9, SEQ ID NO: 10, or SEQ ID NO: 11;
a gene comprising a promoter sequence comprising SEQ ID NO: 12, SEQ ID NO: 13, SEQ ID NO: 14, SEQ ID NO: 15, or SEQ ID NO: 16;
a gene comprising a nucleotide sequence that encodes a CCA-adding enzyme having a deletion, a substitution, or an insertion of at least one amino acid when compared to SEQ ID NO: 2 or SEQ ID NO: 7;
a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11;
a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; and
a gene comprising a promoter sequence having at least 80% sequence identity to SEQ ID NO: 12, SEQ ID NO: 13, SEQ ID NO: 14, SEQ ID NO: 15, or SEQ ID NO: 16.

2. A S. lycopersicum plant comprising a modified CCA gene of paragraph 1, wherein the deletion, substitution, or insertion of at least one amino acid is present in a conserved domain or an active site of the encoded CCA-adding enzyme.

3. A S. lycopersicum plant comprising a modified CCA gene of paragraph 1 comprising a modification in the promoter sequence, which promoter sequence comprises SEQ ID NO: 3, in particular a modification in a regulatory sequence of the promoter sequence.

4. A S. lycopersicum plant comprising a modified CCA gene of paragraph 3, wherein the modification in the promoter sequence comprises a deletion.

5. A S. lycopersicum plant comprising a modified CCA gene of paragraph 4, wherein the deletion in the promoter sequence comprises the deletion of SEQ ID NO: 4.

6. A S. lycopersicum plant of any of the paragraphs 1-5, comprising a combination of two or more modifications in one CCA gene, in particular a combination of a modification in the promoter sequence and a modification in the coding sequence.

7. A S. lycopersicum plant comprising a modified CCA gene of any of the paragraphs 1-5, wherein a CCA-adding enzyme having at least 80% sequence identity to SEQ ID NO: 8 comprises at least one of a N535D substitution, an R553S substitution, or a K579N substitution; a CCA-adding enzyme having at least 80% sequence identity to SEQ ID NO: 9 comprises at least one of a K450E substitution, a R553S substitution, or a K579N substitution; a CCA-adding enzyme having at least 80% sequence identity to SEQ ID NO: 10 comprises at least one of a K316N substitution or a A317V substitution; a CCA-adding enzyme having at least 80% sequence identity to SEQ ID NO: 11 comprises at least a C211R substitution; or a CCA-adding enzyme having at least 80% sequence identity to SEQ ID NO: 8, SEQ ID NO: 9, SEQ ID NO: 10, or SEQ ID NO: 11 comprises any one of those substitutions on the corresponding position of a homologous sequence.

8. A S. lycopersicum plant comprising a modified CCA1 gene as defined in any of the paragraphs 1-7.

9. A S. lycopersicum plant of paragraph 8, wherein the plant comprises the modified CCA1 gene homozygously.

10. A S. lycopersicum plant of any of the paragraphs 1-7, wherein a modified CCA gene is as comprised in the genome of a S. lycopersicum plant representative seed of which was deposited with the NCIMB under deposit number NCIMB 42882, NCIMB 42885, NCIMB 42887, NCIMB 42890, NCIMB 43511, or NCIMB 43512.

11. A S. lycopersicum plant of paragraph 8 or 9, wherein a modified CCA1 gene is as comprised in the genome of a S. lycopersicum plant representative seed of which was deposited with the NCIMB under deposit number NCIMB 42882, NCIMB 42885, NCIMB 42887, NCIMB 42890, NCIMB 43511, or NCIMB 43512.

12. Seed, wherein a plant grown from the seed comprises a modified CCA gene as defined in any of the paragraphs 1-7, or 10.

13. Use of a marker for identification of a modified CCA gene, which comprises the identification of a modification of an A to T SNP on position 948 of SEQ ID NO: 1; a C to T SNP on position 950 of SEQ ID NO: 1; an A to G SNP on position 1348 of SEQ ID NO: 1; an A to G SNP on position 1603 of SEQ ID NO: 1; an A to T SNP on position 1659 of SEQ ID NO: 1; a G to T SNP on position 1737 of SEQ ID NO: 1; a T to C SNP on position 631 of SEQ ID NO: 5; or for identification of a deletion in SEQ ID NO: 3 comprising SEQ ID NO: 4; or for identification of a modification on a corresponding position of a homologous sequence having at least 80% sequence identity to SEQ ID NO: 1 or SEQ ID NO: 3.

14. Use of a marker of paragraph 13 for identification of ToBRFV resistance in a Solanum lycopersicum plant or in a Solanum pimpinellifolium plant.

15. Method for producing a ToBRFV resistant S. lycopersicum plant, comprising introducing a modification as defined in any of the paragraphs 1-7 in a CCA gene.

16. Method for producing a ToBRFV resistant S. lycopersicum plant, comprising introducing a modified CCA gene as defined in any of the paragraphs 1-7, or 10, from a S. lycopersicum or S. pimpinellifolium source plant.

17. Method for selecting a ToBRFV resistant S. lycopersicum plant, comprising identifying the presence of a modification in a CCA gene, optionally testing the plant for ToBRFV resistance, and selecting a plant that comprises said modification as a ToBRFV resistant plant.

18. Method of paragraph 17, wherein the identification of the modified CCA gene is performed by using a marker as defined in paragraph 13.

19. Method for the production of a S. lycopersicum plant which is resistant to ToBRFV, said method comprising:
a) crossing a first S. lycopersicum or S. pimpinellifolium parent plant comprising a modified CCA gene, of any of the paragraphs 1-7, or 10, with a second, S. lycopersicum, parent plant;
b) optionally performing one or more rounds of selfing and/or crossing of the plant resulting from the cross in step a) to obtain a further generation population;
c) selecting from the plant resulting from the cross in step a), or from the further generation population of step b), a plant that comprises the modified CCA gene, wherein the selected plant is resistant to ToBRFV.

20. Method of paragraph 19, wherein the second parent plant also comprises a modified CCA gene.

21. Method of paragraph 19, wherein selection of a plant comprising a modification in a CCA gene is performed by using a marker of paragraph 13.

22. Method of paragraph 19, wherein a plant which is resistant to ToBRFV is phenotypically selected, by using a bio-assay for ToBRFV resistance.

23. Method of paragraph 19, wherein the plant used as a first parent plant is a plant grown from seed deposited under NCIMB Accession number NCIMB 42882, NCIMB 42885, NCIMB 42887, NCIMB 42890, NCIMB 43511, or NCIMB 43512, or is a progeny plant of any of these deposits that has retained the modified CCA gene.

24. Method for the production of hybrid S. lycopersicum seed comprising crossing a first parent plant with a second parent plant and harvesting the resultant hybrid seed, wherein the first parent plant and/or the second parent plant is a plant comprising a modified CCA gene of any of the paragraphs 1-7, or 10, and wherein the presence of said modified CCA gene leads to ToBRFV resistance in a plant that is grown from the hybrid seed.

25. Method for the production of hybrid S. lycopersicum seed, comprising crossing a first parent plant with a second parent plant and harvesting the resultant hybrid seed, wherein the first parent plant and/or the second parent plant is a plant obtained by the method of paragraph 19.

26. A hybrid S. lycopersicum plant that is resistant to ToBRFV, which is obtained by the method of paragraph 24 or 25.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A Solanum lycopersicum plant comprising a modified CCA gene on chromosome 11 that leads to Tomato Brown Rugose Fruit Virus (ToBRFV) resistance, wherein the modified CCA gene comprises:
a gene comprising a nucleotide sequence that encodes a CCA-adding enzyme according to SEQ ID NO: 8, SEQ ID NO: 9, SEQ ID NO: 10, or SEQ ID NO: 11;
a gene comprising a promoter sequence comprising SEQ ID NO: 12, SEQ ID NO: 13, SEQ ID NO: 14, SEQ ID NO: 15, or SEQ ID NO: 16;
a gene comprising a nucleotide sequence that encodes a CCA-adding enzyme having a deletion, a substitution, or an insertion of at least one amino acid when compared to SEQ ID NO: 2 or SEQ ID NO: 7;
a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11;
a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; or
a gene comprising a promoter sequence having at least 80% sequence identity to SEQ ID NO: 12, SEQ ID NO: 13, SEQ ID NO: 14, SEQ ID NO: 15, or SEQ ID NO: 16.

2. (canceled)

3. The plant of claim 1, wherein the deletion, substitution, or insertion of at least one amino acid is present in a conserved domain or an active site of the encoded CCA-adding enzyme.

4. The plant of claim 1, comprising a modification in the promoter sequence, which promoter sequence comprises SEQ ID NO: 3.

5. The plant of claim 4, wherein the modification is in a regulatory sequence of the promoter sequence; or
the modification comprises a deletion in the promoter sequence.

6. (canceled)

7. The plant of claim 5, wherein the deletion in the promoter sequence comprises the deletion of SEQ ID NO: 4.

8. The plant of claim 1, comprising a combination of two or more modifications in one CCA gene,
wherein the two or more modifications is a modification in the promoter sequence and a modification in the coding sequence.

9. (canceled)

10. The plant of claim 1, wherein
the CCA-adding enzyme having at least 80% sequence identity to SEQ ID NO: 8 comprises at least one of a N535D substitution, an R553S substitution, or a K579N substitution;
the CCA-adding enzyme having at least 80% sequence identity to SEQ ID NO: 9 comprises at least one of a K450E substitution, a R553S substitution, or a K579N substitution;
the CCA-adding enzyme having at least 80% sequence identity to SEQ ID NO: 10 comprises at least one of a K316N substitution or a A317V substitution;
the CCA-adding enzyme having at least 80% sequence identity to SEQ ID NO: 11 comprises at least a C211R substitution; or
the CCA-adding enzyme having at least 80% sequence identity to SEQ ID NO: 8, SEQ ID NO: 9, SEQ ID NO: 10, or SEQ ID NO: 11 comprises any one of those substitutions at the corresponding position of a homologous sequence.

11. (canceled)

12. The plant of claim 1, wherein the plant comprises the modified CCA1 gene homozygously.

13. The plant of claim 1, wherein a modified CCA gene is as comprised in the genome of a Solanum lycopersicum plant representative seed of which was deposited with the NCIMB under deposit number NCIMB 42882, NCIMB 42885, NCIMB 42887, NCIMB 42890, NCIMB 43511, or NCIMB 43512.

14. (canceled)

15. A Solanum lycopersicum seed from, or that produces the Solanum lycopersicum plant of claim 1.

16. A method for selecting a Solanum lycopersicum plant comprising a modified CCA gene comprising:
identifying in the genome of a Solanum lycopersicum plant:
a modification of an A to T SNP at position 948 of SEQ ID NO: 1; a C to T SNP at position 950 of SEQ ID NO: 1; an A to G SNP at position 1348 of SEQ ID NO: 1; an A to G SNP at position 1603 of SEQ ID NO: 1; an A to T SNP a position 1659 of SEQ ID NO: 1; a G to T SNP at position 1737 of SEQ ID NO: 1; a T to C SNP at position 631 of SEQ ID NO: 5; or
a deletion in SEQ ID NO: 3 comprising SEQ ID NO: 4; or
a modification at a corresponding position of a homologous sequence having at least 80% sequence identity to SEQ ID NO: 1 or SEQ ID NO: 3;
thereby selecting the Solanum lycopersicum plant comprising the modified CCA gene, wherein the presence of said modified CCA gene leads to ToBRFV resistance as compared to a plant lacking the modified CCA gene.

17. (canceled)

18. A method for producing a ToBRFV resistant Solanum lycopersicum plant, comprising:
introducing a modification into a CCA gene into a Solanum lycopersicum plant wherein the modification results in the modified CCA gene of the Solanum lycopersicum plant of claim 1; and/or
introducing the modified CCA gene of the Solanum lycopersicum plant of claim 1 from a S. lycopersicum or S. pimpinellifolium source plant into a Solanum lycopersicum plant.

19. (canceled)

20. A method for selecting a ToBRFV resistant Solanum lycopersicum plant, comprising identifying the presence of a modification in a CCA gene, optionally testing the plant for ToBRFV resistance, and selecting the plant that comprises the modification as a ToBRFV resistant plant;
wherein the modified CCA gene comprises:
a gene comprising a nucleotide sequence that encodes a CCA-adding enzyme according to SEQ ID NO: 8, SEQ ID NO: 9, SEQ ID NO: 10, or SEQ ID NO: 11;
a gene comprising a promoter sequence comprising SEQ ID NO: 12, SEQ ID NO: 13, SEQ ID NO: 14, SEQ ID NO: 15, or SEQ ID NO: 16;
a gene comprising a nucleotide sequence that encodes a CCA-adding enzyme having a deletion, a substitution, or an insertion of at least one amino acid when compared to SEQ ID NO: 2 or SEQ ID NO: 7;
a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11;
a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; or
a gene comprising a promoter sequence having at least 80% sequence identity to SEQ ID NO: 12, SEQ ID NO: 13, SEQ ID NO: 14, SEQ ID NO: 15, or SEQ ID NO: 16.

21. A method for producing a Solanum lycopersicum plant resistant to ToBRFV, said method comprising:
a) crossing a first S. lycopersicum or S. pimpinellifolium parent plant comprising the modified CCA gene of the Solanum lycopersicum plant of claim 1, with a second S. lycopersicum parent plant;
b) optionally performing one or more rounds of selfing and/or crossing of the plant resulting from the cross in step a) to obtain a further generation population;
c) selecting from the plant resulting from the cross in step a), or from the further generation population of step b), the plant that comprises a modified CCA gene, wherein the selected plant is resistant to ToBRFV.

22. The method of claim 21, wherein the second parent plant comprises a modified CCA gene.

23. The method of claim 21, further comprising selecting a plant comprising a modified CCA gene, wherein the identifying of the modified CCA gene in the plant is the method of claim 16.

24. The method of claim 21, wherein a plant which is resistant to ToBRFV is phenotypically selected with a bio-assay for ToBRFV resistance.

25. The method of claim 21, wherein the first parent plant is a plant grown from seed deposited under NCIMB Accession number NCIMB 42882, NCIMB 42885, NCIMB 42887, NCIMB 42890, NCIMB 43511, or NCIMB 43512, or is a progeny plant of any of these deposits that has retained the modified CCA gene.

26. A method for producing hybrid Solanum lycopersicum seed comprising
crossing a first Solanum lycopersicum parent plant with a second Solanum lycopersicum parent plant and
harvesting the resultant hybrid seed,
wherein the first Solanum lycopersicum parent plant and/or the Solanum lycopersicum second parent plant comprises a modified CCA gene, and
wherein the presence of said modified CCA gene leads to ToBRFV resistance in a plant that is grown from the hybrid seed;
wherein the modified CCA gene comprises:
a gene comprising a nucleotide sequence that encodes a CCA-adding enzyme according to SEQ ID NO: 8, SEQ ID NO: 9, SEQ ID NO: 10, or SEQ ID NO: 11;
a gene comprising a promoter sequence comprising SEQ ID NO: 12, SEQ ID NO: 13, SEQ ID NO: 14, SEQ ID NO: 15, or SEQ ID NO: 16;
a gene comprising a nucleotide sequence that encodes a CCA-adding enzyme having a deletion, a substitution, or an insertion of at least one amino acid when compared to SEQ ID NO: 2 or SEQ ID NO: 7;
a gene comprising a nucleotide sequence that encodes a CCA-adding enzyme having a deletion, a substitution, or an insertion of at least one amino acid when compared to SEQ ID NO: 8, SEQ ID NO: 9, SEQ ID NO: 10, or SEQ ID NO: 11;
a gene comprising a nucleotide sequence that encodes a CCA-adding enzyme having at least 80% sequence identity to SEQ ID NO: 8, SEQ ID NO: 9, SEQ ID NO: 10, or SEQ ID NO: 11; or
a gene comprising a promoter sequence having at least 80% sequence identity to SEQ ID NO: 12, SEQ ID NO: 13, SEQ ID NO: 14, SEQ ID NO: 15, or SEQ ID NO: 16;
or the method comprises:
crossing a first Solanum lycopersicum parent plant with a second Solanum lycopersicum parent plant and
harvesting the resultant hybrid seed,
wherein the first Solanum lycopersicum parent plant and/or the second Solanum lycopersicum parent plant is the plant obtained by the method of claim 21.

27. (canceled)

28. A hybrid S. lycopersicum plant that is resistant to ToBRFV obtained by growing the hybrid seed obtained by the method of claim 26.

29. (canceled)


SEQUENCE LISTING (19 sequences)

SEQ ID NO: 1 - DNA, 1770 bp, Solanum lycopersicum
  misc_feature 1..1770: note = CCA1 cDNA|Sl3_00 Solanum lycopersicum (S) wildtype
  source 1..1770: mol_type = genomic DNA
atgaaggctttgagtatagccgtagcagcattttcaacattccatacaactcgccctcgtctcttcactttcccaaacccttctacttcttcttctctccttcttccgtccttcaaactaaagagattttctatcttctccattcaacaaccgccgccggcgttagggttttacgccggagttacaacaatggtggagggctcagcatttcagccgttgtcagttcaagtgaaggaccatattgatttatcgccaaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccagtgcaatcccgatcagtcaaaacacttggaaacagcgaggatgcgccttttcgacgtatggattgattttgtcaacttaagagcagaagactacagtgagaacagtcgaattccaacaatgaaatttgggactgcaaaacaggatgctgatcgcagggatttaaccattaatagcttgttttacaacattaacaccagatcagtcgaagatcttacagaaagaggcattgcagatctgaagtctggaaaaattgtcactccactacctccaaagcagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgttacttgcttgggttcagcttgggaacttttaaaccttattggacgctcctcattttcagatgatcagaggagactttgcctgtatgctgctttatttttgccattcagagagaccatttatagagataacaaagccaaaaagattccagttgtcaattatatttttcgcaattctctcaagttgaaggccagtgatgcggaaatagtcatgagcttgcatacggtaactaggaaatttgtcaccttaatatcactactcatttctaaagacgatatccaagttcttgaagttgattggaaaagggataccattgaggttccaatagcttcaaaacttagaatattgactggtttgctacttcgagaaattaaagaattttggcgtgtcgctcttcttctatctatgctgctacatcctgttgacgttgtgtcctctacaagtttctcaaatgagaactttgaactggacaagagtagcggattatttaagacagtagagaatgcagtaagaacactaggtctggagaaggtttgggaaatgaaaccactggttaatggaaaggaaattatgaatattttggagattaaaagtggagggccagttgttagagaatggcaacaaaaactgctggagtggcagctcgcacacccttctggttcagctgaagaatgcatcgactggatgaagcaagcacagtcaaaacgtgcaagaaccgagtag

SEQ ID NO: 2 - protein, 589 aa, Solanum lycopersicum
  REGION 1..589: note = CCA1 protein|Wildtype CCA-adding enzyme
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVREWQQKLLEWQLAHPSGSAEECIDWMKQAQSKRARTE

SEQ ID NO: 3 - DNA, 1001 bp, Solanum lycopersicum
  misc_feature 1..1001: note = Promoter of the wildtype CCA1 gene
  source 1..1001: mol_type = genomic DNA
atatactatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatttaggtatgcctcaagttacgtgatttccaatagttttacaacaataataatacaaatataaatcattctttccattttttttgtctttctttctctcactttatacaaacacaaattgtgtaaaaaattatacaaaatttgtgtttgtataaaacgagagagagatttcatatacaaatattttaacttgattcaattgtatacgagtttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttatttttttttaaatatttatttttttttaaataaaaaaataaacttgccacatcatgaaagatgtagttgggatgacccgacccgttttctatgcttatgagagcgggtcatcacttgtccgttagaaagagagaagaa

SEQ ID NO: 4 - DNA, 10 bp, Solanum lycopersicum
  misc_feature 1..10: note = Deletion in the promoter sequence
  source 1..10: mol_type = genomic DNA
atatttattt

SEQ ID NO: 5 - DNA, 1101 bp, Solanum lycopersicum
  misc_feature 1..1101: note = CCA2 cDNA|Sl3_00 Solanum lycopersicum (S) wildtype
  source 1..1101: mol_type = genomic DNA
atgaaggctttgagtatagccgtagcagcattttcaacattccatacaactcgccctcgtctcttcactttcccaaacccttctacttcttcttctctccttcttccgtccttcaaactaaagagattttctatcttctccgttcaacaaacgccgccggcgttagggttttacgccggagttacaacaatggtggagggctcagcatttcagccgttgtcagttcaagtgaaggaccatattgatttatcgccgaaagaggagcagatattcaaccgtcttcttcaagttgtcgagcattataatatgggaacacagcttcgtgttgctggtggttgggttcgcgataagctgctaggtaaagagtgctatgatattgatattgctcttgacaacatgtatggacgagaattctgtgagaaagtgaacgagtacttgtcagcatcaggtgaggaaacccaaggagttggtgttatccaatgcaatcctgatcagtcaaaacacttggaaacagcaaggatgcgcctttccgacgtatggattgattttgtcaacttaagagcagaagactacagtgagaacagtcgaattccaacaatgaaatttggcactgcaaaacaggatgctgattgcagggatttaaccatcaatagcttgttttacaacattaacaccagatcagtcgaagatcttacagaaagaggcattgcagatctgaagtctggaaaaattgtcactccactacctccaaaacagacctttttggatgatcccctaagagttcttcgagctattcgttttggtgcaaggtttgggtttatattagatgaagaactaaagaaggcagctgcagatgaagacgtgagagttgctattgctgataaaattagcagagaacgcataggacatgaaattgatcttatgatgtctggtgaccaaccggtcaaagcaatgacttatattgctgacttgaagttattttcggctgtatttagtcttccttctgattttgagccatcaattcctgatggatgtgataggctgtgtgtttcttgcttgggaacttttaaaccttattggacgctcctcattttcagatga

SEQ ID NO: 6 - DNA, 11 bp, Solanum lycopersicum
  misc_feature 1..11: note = Deletion in a CCA gene
  source 1..11: mol_type = genomic DNA
ttcagcttggg

SEQ ID NO: 7 - protein, 366 aa, Solanum lycopersicum
  REGION 1..366: note = CCA2 protein|Wildtype CCA-adding enzyme
MKALSIAVAAFSTFHTTRPRLFTFPNPSTSSSLLLPSFKLKRFSIFSVQQTPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLSDVWIDFVNLRAEDYSENSRIPTMKFGTAKQDADCRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVSCLGTFKPYWTLLIFR

SEQ ID NO: 8 - protein, 589 aa, Solanum lycopersicum
  REGION 1..589: note = CCA1 protein|NCIMB43511, NCIMB43512
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWKRDTIEVPIASKLRILTGLLLREIKEFWRVALLLSMLLHPVDVVSSTSFSNENFELDKSSGLFKTVENAVRTLGLEKVWEMKPLVDGKEIMNILEIKSGGPVVSEWQQKLLEWQLAHPSGSAEECIDWMNQAQSKRARTE

SEQ ID NO: 9 - protein, 589 aa, Solanum lycopersicum
  REGION 1..589: note = CCA1 protein|TO1
MKALSIAVAAFSTFHTTRPRLFTFPNPSTSSSLLLPSFKLKRFSIFSIQQPPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLFDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVTCLGSAWELLNLIGRSSFSDDQRRLCLYAALFLPFRETIYRDNKAKKIPVVNYIFRNSLKLKASDAEIVMSLHTVTRKFVTLISLLISKDDIQVLEVDWERDTIEVPIASKLRILTGLLLREIKEFWRVALLLSMLLHPVDVVSSTSFSNENFELDKSSGLFKTVENAVRTLGLEKVWEMKPLVNGKEIMNILEIKSGGPVVSEWQQKLLEWQLAHPSGSAEECIDWMNQAQSKRARTE

SEQ ID NO: 10 - protein, 366 aa, Solanum lycopersicum
  REGION 1..366: note = CCA2 protein|NCIMB43512
MKALSIAVAAFSTFHTTRPRLFTFPNPSTSSSLLLPSFKLKRFSIFSVQQTPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLSDVWIDFVNLRAEDYSENSRIPTMKFGTAKQDADCRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVNVMTYIADLKLFSAVFSLPSDFEPSIPDGCDRLCVSCLGTFKPYWTLLIFR

SEQ ID NO: 11 - protein, 366 aa, Solanum lycopersicum
  REGION 1..366: note = CCA2 protein|TO1
MKALSIAVAAFSTFHTTRPRLFTFPNPSTSSSLLLPSFKLKRFSIFSVQQTPPALGFYAGVTTMVEGSAFQPLSVQVKDHIDLSPKEEQIFNRLLQVVEHYNMGTQLRVAGGWVRDKLLGKECYDIDIALDNMYGREFCEKVNEYLSASGEETQGVGVIQCNPDQSKHLETARMRLSDVWIDFVNLRAEDYSENSRIPTMKFGTAKQDADRRDLTINSLFYNINTRSVEDLTERGIADLKSGKIVTPLPPKQTFLDDPLRVLRAIRFGARFGFILDEELKKAAADEDVRVAIADKISRERIGHEIDLMMSGDQPVKAMTYIADLKLFSAVFSLPSDFEPSIPDGCDRLCVSCLGTFKPYWTLLIFR

SEQ ID NO: 12 - DNA, 1000 bp, Solanum lycopersicum
  misc_feature 1..1000: note = CCA1 promotor|NCIMB43511, NCIMB43512
  source 1..1000: mol_type = genomic DNA
atggtatgaaatttaccatatacatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatataggtatgcctcaagttacgtgatttccaatagttttacaacaataataatacaaatataaatcattctttccatcttttttgtctttctttctctcactttatacaaacacaaattgtgtaaaaaattatacaaaatttgtgtttgtataaaacgagagagagatttcataaacaaatattttaacttgattcaattgtatacgaatttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttattttttttttaaataaaaaaataaacttgccacatcatgaaagatgtagttgggatgacccgacccgttttctatgcttatgagagcgggtcatcacttgtccgttagaaagagagaagaa

SEQ ID NO: 13 - DNA, 1001 bp, Solanum lycopersicum
  misc_feature 1..1001: note = CCA1 promotor|TO1
  source 1..1001: mol_type = genomic DNA
aatggtatgaaatttaccatatacaatatctatatatattactttggtgtgttaaaagagtgagaatgagatattacatcaaaataatttttcttccttatttatttgtttggtttcttaagtaaaatatatattttttgttaaattctgatttctagttatagtacaagaaaaacttgttgaatacacctttatcggtgaaatatccttaaggacagtgatataggtatgcctcaagttacgtgatttccaatagttttacaacaataataatacaaatataaatcattctttccatcttttttgtctttctttctctcactttatacaaacacaaattgtgtaaaaaattatacaaaatttgtgtttgtataaaacgagagagagatttcataaacaaatattttaacttgattcaattgtatacgaatttaaagtttatgcagatatacaaactgaatcattcataaatatacatagtagttagatatatacaattatctaattgatatacatatacaattcacctctctcccctctctatcccactctagctcgcctctctcatctctctctctcaatcttgctcgtcgctctaacctaacacagagaacatatacatatacaaacacaatattcatagacacagacgaatgagcgctcgattaatataaattggcgcgatttatacaaattagatgctccataacaaacataatctgattatgaagcataaataacaaaattatagctgagaactctaatataaatttttttttatctttgatacacctaaaatttactcaataaaatgtgactatatttattctaagctaattacattattgtacttattcatgttttaactttaactatttttatttttttttaaataaaaaaataaacttgccacatcatgaaagatgtagttgggatgacccgacccgttttctatgcttatgagagcgggtcatcacttgtccgttagaaagagagaagaa

SEQ ID NO: 14 - DNA, 1002 bp, Solanum lycopersicum
  misc_feature 1..1002: note = CCA2 promotor|NCIMB43511
  source 1..1002: mol_type = genomic DNA
aagactgaagggtatggaatttaccatatactatatctatatatattactttggtttgttaaaagagggacaatgagatattacatcaaaataatttttcttttcttccttatttatttatttggtttcttaagtaaaatatatttttttgttagattctttattcctaattttagtacaagaaaaacttgttgaatacacatttactggtgaaatatccttaaggataatgacataggcacgcctcaagttacgtgacttccaatagttttacaacaataattatacaaatataaatatttcttgccatctttttttgtctctctctcactttatacaaacacaagttatacaaaagacaatataaaatttgtgtttgtataaaacgagagagagatttcatatacaaatgttttaacttgattcaattatatacgaatttaaattttatgtagatatatagactgaatcattcattaatatacatagtagttaaatatatacaattatctaatcggtatacatatacaattcacctctctccactctctacccactctagctcgtctctctcatctctctctctcaatctcgctcgtcgctctaaccaaacacagagaacatatacatatacaaacacaatttcatagacacagacgaatgagcgctcgattaatacaattggagcgatttatacaaattagatgctccataacaaacataatctggttatgaagcgtaaataacaaaattatagctgtagagctctaatataattttttatctttgatactcctaaaatttactcaataaaatgtgactatatttattctaagctaattacattattgtatttattcatgttttaactttaactatatatttttttaaaaataaaaaaataaacttgccacatcatgaaagatgtggttgagatgacccgacccgtttcttatgcttatgagagcgggtcatcacttgtccgttagaaagagagaagaa

SEQ ID NO: 15 - DNA, 1002 bp, Solanum lycopersicum
  misc_feature 1..1002: note = CCA2 promotor|NCIMB43512
  source 1..1002: mol_type = genomic DNA
aagactgaagggtatggaatttaccatatactatatctatatatattactttggtttgttaaaagagggacaatgagatattacatcaaaataatttttcttttcttccttatttatttatttggtttcttaagtaaaatatatttttttgttagattctttattcctaattttagtacaagaaaaacttgttgaatacacatttactggtgaaatatccttaaggataatgacataggcacgcctcaagttacgtgacttccaatagttttacaacaataattatacaaatataaatatttcttgccatctttttttgtctctctctcactttatacaaacacaagttatacaaaagacaatataaaatttgtgtttgtataaaacgagagagagatttcatatacaaatgttttaacttgattcaattatatacgaatttaaattttatgtagatatatagactgaatcattcattaatatacatagtagttaaatatatacaattatctaatcggtatacatatacaattcacctctctccactctctacccactctagctcgtctctctcatctctctctctcaatctcgctcgtcgctctaaccaaacacagagaacatatacatatacaaacacaatttcatatacacagacgaatgagcgctcgattaatacaattggagcgatttatacaaattagatgctccataacaaacataatctggttatgaagcgtaaataacaaaattatagctgtagagctctaatataattttttatctttgatactcctaaaatttactcaataaaatgtgactatatttattctaagctaattacattattgtatttattcatgttttaactttaactatatatttttttaaaaataaaaaaataaacttgccacatcatgaaagatgtggttgagatgacccgacccgtttcttatgcttatgagagcgggtcatcacttgtccgttagaaagagagaagaa

SEQ ID NO: 16 - DNA, 988 bp, Solanum lycopersicum
  misc_feature 1..988: note = CCA2 promotor|TO1
  source 1..988: mol_type = genomic DNA
aagactgaagggtatggaatttaccatatactatatctatatatattactttggtttgttaaaagagggacaatgagatattacatcaaaataatttttcttttcttccttatttatttatttggtttcttaagtaaaatatattttttgttagattctttattcctaattttagtacaagaaaaacttgttgaatacacatttactggtgaaatatccttaaggataatgacataggcacgcctcaagttacgtgacttccaatagttttacaacaataattatacaaatataaatatttcttgccatctttttttgtctctctctcactttatacaaacacaagttatacaaaagacaatataaaatttgtgtttgtataaaacgagagagagatttcatatacaaatgttttaacttgattcaattatatacgaatttaaattttatgcagatatatagactgaatcattcattaatatacatagtagttaaatatatacaattatctaatcggtatacatatacaattcacctctctccactctctacccactctagctcgtctctctcaatctcgctcgtcgctctaaccaaacacagaaaacatatacatatacaaacacaatttcatagacacagacgaatgagcgctcgattaatacaattggagcgatttatacaaattagatgctccataacaaacataatctggttatgaagcgtaaataacaaaattatagctgtagagctctaatataattttttatctttgatactcctaaaatttactcaataaaatgtgactatatttattctaagctaattacattattgtatttattcatgttttaactttaactatatatttttttaaaaataaaaaaataaacttgccacatcatgaaagatgtagttgagatgacccgacccgtttcttatgcttatgagagcgggtcatcacttgtccgttagaaagagagaagaa

SEQ ID NO: 17 - DNA, 1001 bp, Solanum lycopersicum
  misc_feature 1..1001: note = Promoter of the wildtype CCA2 gene
  source 1..1001: mol_type = genomic DNA
aagactgaagggtatggaatttaccatatactatatctatatatattactttggtttgttaaaagagggacaatgagatattacatcaaaataatttttcttttcttccttatttatttatttggtttcttaagtaaaatatattttttgttagattctttattcctaattttagtacaagaaaaacttgttgaatatacatttactggtgaaatatccttaaagataatgacataggcacgcctcaagttacgtgacttccaatagttttacaacaataattatacaaatataaatatttcttgccatctttttttgtctctctctcactttatacaaacacaagttatacaaaagacaatataaaatttgtgtttgtataaaacgagagagagatttcatatacaaatgttttaacttgattcaattatatacgaatttaaattttatgcagatatatagactgaatcattcattaatatacatagtagttaaatatatacaattatctaatcggtatacatatacaattcacctctctccactctctacccactctagctcgtctctctcatctctctctctcaatctcgctcgtcgctctaaccaaacacagagaacatatacatatacaaacacaatttcatagacacaaacgaatgagcgctcgattaatacaattggagcgatttatacaaattagatgctccataacaaacataatctggttatgaagcgtaaataacaaaattatagctgtagagctctaatataattttttatctttgatactcctaaaatttactcaataaaatgtgactatatttattctaagctaattacattattgtatttattcatgttttaactttaactatatatttttttaaaaataaaaaaataaacttgccacatcatgaaagatgtagttgagatgacccgacccgtttcttatgcttatgagagcgggtcatcacttgtccgttagaaagagagaagaa

SEQ ID NO: 18 - DNA, 17 bp, Solanum lycopersicum
  misc_feature 1..17: note = Deletion in the promoter sequence
  source 1..17: mol_type = genomic DNA
tttttaaatatttattt

SEQ ID NO: 19 - DNA, 17 bp, Solanum lycopersicum
  misc_feature 1..17: note = Deletion in the promoter sequence
  source 1..17: mol_type = genomic DNA
aaatatttatttttttt